(19) United States
(12) Patent Application Publication
(10) Pub. No.: US 2022/0028488 A1
(43) Pub. Date: Jan. 27, 2022

(54) DRUG DISCOVERY AND EARLY DISEASE IDENTIFICATION PLATFORM USING ELECTRONIC HEALTH RECORDS, GENETICS AND STEM CELLS

(71) Applicant: President and Fellows of Harvard College, Cambridge, MA (US)

(72) Inventors: Lee L. Rubin, Cambridge, MA (US); Isaac S. Kohane, Cambridge, MA (US); Scott Logan Lipnick, Cambridge, MA (US)

(73) Assignee: President and Fellows of Harvard College, Cambridge, MA (US)

(21) Appl. No.: 17/309,522

(22) PCT Filed: Dec. 6, 2019
(86) PCT No.: PCT/US2019/064887

§ 371 Date: Jun. 3, 2021

Related U.S. Application Data
(60) Provisional application No. 62/776,719, filed on Dec. 7, 2018.

(51) Int. Cl. G16B 25/10; G16B 20/40; C12N 5/074; G01N 33/50; G16B 40/00; C12Q 1/6883; G16H 10/60; G16H 10/40; G16H 70/40

(52) U.S. Cl. CPC G16B 25/10; C12Q 2600/156; C12N 5/0696; G01N 33/5008; G01N 33/5073; G16B 40/00; C12Q 1/6883; G16H 10/60; G16H 10/40; G16H 70/40; G01N 2800/28; C12N 2506/45; C12N 2506/1307; G01N 2800/2835; G01N 2800/10; G16B 20/40

(57) ABSTRACT

Disclosed are systems and methods for utilizing electronic health record data (EHR) to group patients with diagnoses of a common disease of interested (e.g. Parkinson's disease) into phenotype clusters that share common clusters of other diagnosis codes (in addition to the Parkinson's diagnosis, for example). The phenotype clusters can be processed with genetic data to identify convergent genetic mutations within each phenotype cluster and output phenotypic-genomic clusters. Then, stem cells from the patients (e.g. iPSCs) may be differentiated into a desired cell type within each phenotypic-genomic cluster, and these differentiated cells may be assayed to identify defects in the various tissues types or organoids. Additionally, the differentiated cells can then be tested with candidate agents to determine whether the agents reverse the defective phenotypes identified in the differentiated tissues or organoids.

### RELATED APPLICATION

This application claims the benefit under 35 U.S.C. § 119(e) of U.S. provisional application No. 62/776,719, filed Dec. 7, 2018, which is incorporated by reference herein in its entirety.

### FIELD

The present invention is directed to the field of drug discovery and disease classification and identification.

### BACKGROUND

The following description includes information that may be useful in understanding the present invention. It is not an admission that any of the information provided herein is prior art or relevant to the presently claimed invention, or that any publication specifically or implicitly referenced is prior art.

Identifying new treatments for genetic related diseases presents a huge challenge for basic and pharmaceutical scientists. For instance, diseases of the nervous system, including neurodegenerative and neuropsychiatric disorders, have much genetic and phenotypic variability. As a group, neurodegenerative diseases, such as Alzheimer's disease (AD) and PD, are driven by rare penetrant genes, but are also associated with numerous common gene variants (Lambert et al., 2013; Nalls et al., 2014). In addition, they are heavily influenced by environmental factors and other complicated processes such as aging. Neuropsychiatric diseases, such as schizophrenia, are associated with hundreds of common gene variants, each one of which increases the probability of getting the disease by a small percentage (Pardiñas et al., 2018). This means that each individual disorder likely has many forms that may overlap partially, but not completely. For example, PD is associated with death of midbrain dopaminergic (DA) neurons, but some forms involve cognitive loss, and only some are associated with the formation of Lewy bodies. Similarly, all patients with AD have plaques and tangles, but some have early onset (familial) forms while the vast majority have later onset forms associated with aging itself and other genetic risk factors. Accordingly, each of these diseases has many forms and diagnosing and treating them is quite challenging.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 depicts an example of a diagram of an overview of the disclosed platform;

FIG. 2 depicts an example of a flow chart depicting a process for drug discovery;

FIG. 3 depicts an example of a flow chart depicting a process for treating a patient identified as belonging to a sub-class of a disease;

FIG. 4 depicts an example schematic of patent selection and grouping;

FIG. 5 depicts a table illustrating Cohort details for Example 2;

FIG. 6 depicts a graph illustrating Manhattan plots of phenotypes with increased prevalence in SMA for Example 2;

FIG. 7 depicts a table illustrating grouped phenotypes with increased prevalence for SMA patients by group for Example 2;

FIGS. 8A-8C depict bar graphs illustrating the temporal trajectories of grouped phenotypes for Example 2. FIG. 8A represent data from infantile SMA patients in Group 1. FIG. 8B represents data from adolescent onset SMA patients in Group 2; FIG. 8C represent data from adult onset SMA in Group 3;

FIGS. 9A-B depict graphs illustrating Manhattan plots of phenotypes with increased prevalence for SMA for Group 1 in Example 2. FIG. 9A illustrates phenotypes with increased prevalence of SMA compared to controls though lifetime and FIG. 9B illustrates phenotypes with increased prevalence of SMA compared to controls for only data prior to their first Neuromuscular diseases diagnosis where SMN decrease is more likely to drive phenotypes; and

FIGS. 10A-10B depict graphs illustrating Manhattan plots of phenotypes with increased prevalence in SMA for Group 3 in Example 2. FIG. 10A illustrates phenotypes with increased prevalence of SMA compared to controls though lifetime and FIG. 10B illustrates phenotypes with increased prevalence of SMA compared to controls for only data prior to their first Neuromuscular diseases diagnosis where SMN decrease is more likely to drive phenotypes.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Overview

Identifying new treatments for genetic related diseases presents a huge challenge for basic and pharmaceutical scientists. For instance, diseases of the nervous system, including neurodegenerative and neuropsychiatric disorders, have much genetic and phenotypic variability. As a group, neurodegenerative diseases, such as Alzheimer's disease (AD) and PD, are driven by rare penetrant genes, but are also associated with numerous common gene variants (Lambert et al., 2013; Nalls et al., 2014). In addition, they are heavily influenced by environmental factors and other complicated processes such as aging. Neuropsychiatric diseases, such as schizophrenia, are associated with hundreds of common gene variants, each one of which increases the probability of getting the disease by a small percentage (Pardiñas et al., 2018). This means that each individual disorder likely has many forms that may overlap partially, but not completely. For example, PD is associated with death of midbrain dopaminergic (DA) neurons, but some forms involve cognitive loss, and only some are associated with the formation of Lewy bodies. Similarly, all patients with AD have plaques and tangles, but some have early onset (familial) forms while the vast majority have later onset forms associated with aging itself and other genetic risk factors. Accordingly, each of these diseases has many forms and diagnosing and treating them is quite challenging. For instance, neurological and neuropsychiatric diseases are particularly difficult to understand and treat, explaining why there have been few breakthrough medicines in many years. One of the main reasons for this is that each individual CNS disease, while characterized by some shared symptoms and pathology, is quite heterogeneous, meaning that effective treatments may work only on subsets of patients.

Thus, the inventor(s) have combined genome sequencing, electronic health records (EHRs) and induced pluripotent stem cell (iPSC) biology together in a new way to allow for identifying, stratifying, and treating these patients. The inventor(s) have demonstrated the viability of integrating these three technologies through an exploration of how variants known to lead to increased risk of disease result in detectable and relatable phenotypes in vivo (EHR) and in vitro (iPSC-derived). Because genes known to underlie CNS disease, for instance, are expressed in multiple non-neural tissues, the investigation will include non-neural aspects of these diseases.

This raises the important question: is it better to treat the shared symptomatic components of the disease or to divide patients into more specific subclasses that reflect common underlying biological processes driving those disease subclasses? Oncologists facing this decision have clearly opted for the more individualized approach. However, cancers are different from CNS disease in that single druggable driver mutations have been identified in biopsied tumors and now form the basis for much of modern cancer therapy. This type of thinking has not really penetrated into the CNS space and other spaces with genetically complex mechanisms where most treatments are still focused on shared phenotypes rather than more individualized therapies. Thus, this approach leverages advances in genetics, clinical database interrogation, and human stem cell derived models to subdivide disorders (e.g. neural) into distinguishable classes that allows for the development of new, more effective, drugs.

Genomic sequencing is also very important to this approach. Sequence information from a sufficiently large number of diseased patients may lead to some mechanistic clarity—shared converging druggable pathways—that are broadly applicable to a large percentage of patients. However, because each neural disease (and other complex genetic disease) is complicated—PD itself has more than 10 different “monogenic” forms—it is also possible that treating shared pathways is neither optimal nor even possible.

However, additional, surprising, insights can be gained by examining patients' EHR. For example, while it has been known for years that constipation is an early symptom of PD, it now appears that LRRK2, a PD risk gene, is also associated with an increased risk for Crohn's disease, at least in certain populations (Hui et al., 2018). Even more interestingly, a recent report suggests that it is possible to cluster patients by their phenomes (their entire set of medical symptoms), thereby gaining additional data concerning the genetics of symptom susceptibility (Bastarache et al., 2018).

Genome sequencing and EHR provide datasets concerning patients with disease. However, given the disclosed invention, an experimental system capable of generating large numbers of differentiated cells from specific patients will not be required to fully utilize that data, especially for complex polygenic diseases where many common variants combine to dictate disease status. Rather, iPSCs can be used as cellular “avatars” of patients who are fully characterized by a combination of genomic sequencing and long term EHR documentation. Of all the human experimental systems available, this is the single one that is scalable, individualized and capable of generating multiple types of specific tissues.

Further support for these concepts is found in studies of children with Spinal Muscular Atrophy (SMA), a childhood monogenic, motor neuron (MN) disease, caused by a mutation in the SMN1 gene. In those cases, the inventor(s) produced iPSCs from children with severe and milder forms of the disease and derived MNs from each of the lines. The inventors(s) were first able to show that MNs made from these iPSCs indeed were defective, dying much more rapidly than those from unaffected children (Ng et al., 2015; Rodriguez-Muela et al., 2017). Unexpectedly, however, using a combination of nanostring analysis and directed differentiation methods, the inventor(s) also found that the iPSCs themselves were abnormal in their ability to produce endodermal tissues, cardiac muscle and skeletal muscle. In fact, endodermal cells were actually the most sensitive to reduced levels of SMN.

Initially, the interpretation of those results was not altogether clear. Did they indicate that children with SMA might have affected tissues other than MNs? SMN1 is ubiquitously expressed, making this seem at least possible (Lonsdale et al., 2013). Together with the Kohane group at HMS, the inventor(s) referenced an Aetna insurance database of approximately 60 million EHRs to determine if real-world clinical evidence exists for non-neuromuscular phenotypes in SMA patients.

The results, derived from records of about 1000 children with SMA, showed that they have a variety of other significant medical issues including cardiovascular, metabolic, and intestinal defects. The inventor(s) further analyzed a set of children with milder forms of the disease (onset >2 years old) to characterize the SMA disease trajectory and discovered that many of these conditions predate any neuromuscular problems (Lipnick et al., submitted). This means that not only is SMA a multi-system disorder, as the iPSC work predicted, but that important information can be gleaned from EHRs corroborating novel findings from iPSCs. Now that there is an SMA treatment—Spinraza, an intrathecally delivered antisense oligonucleotide that acts only on spinal cord neurons—the recognition that other defects exist has become therapeutically important. In summary, it appears that iPSCs may, in fact, capture features of disease that were both already known and were previously unsuspected. This helps validate their use in exploring the nature of complex neural disorders as described below.

For instance, these findings could be extended to explore Parkinson's disease (PD), as one example—a much more common and complex late-onset neurodegenerative disease. For instance, one could use iPSCs carrying PD-associated mutations as in vitro human surrogates to compare against EHR data from individuals already sequenced at Partners Healthcare System, some of which have the same variants as the iPSCs. LRRK2, one candidate PD gene, is expressed at particularly high levels (much higher than in brain) in human tissues of endodermal origin, especially the lung. Interestingly, LRRK2 inhibitors have known side effects in lung and kidney (also, in large part, an endodermal tissue; Fuji et al., 2015). The PARK2 gene, on the other hand, is expressed at highest levels in brain, muscle, heart and testis, while GBA, another PD risk gene, is highest in the adrenal gland, pituitary, and cardiovascular system (Lonsdale et al., 2013). This raises the possibility that each individual PD-gene mutation could be associated with different defects in non-neural tissues that have mostly gone unrecognized. Thus, one could evaluate both EHR data and data generated using iPSCs to explore these potential non-neuronal phenotypes as described herein.

### SUMMARY

Thus, the inventor(s) have discovered that many genetic diseases exhibit numerous numbers of sub-forms that may have their own genetic underlying mechanisms and environmental factors and may exhibit different phenotypes, including different disease maladies in patients. However, genetics alone has not been sufficient to group patients into disease sub-forms and identify common druggable targets, as it is not clear in those cases which of the common genetic pathways are causing the phenotypes associated with the diseases (unlike in diseases like certain types of cancer that have a single, druggable mutation). Accordingly, the inventor(s) have utilized electronic health record data (EHR) to group patients with diagnoses of a common disease of interested (e.g., Parkinson's disease) into phenotype clusters that share common clusters of other diagnosis codes (in addition to the Parkinson's diagnosis, for example). These result in different phenotype clusters for the disease. It is not required that the genetic cause for the disease (e.g., the genetic mutation(s) that result in onset of the disease) be the same or similar within one phenotype cluster. For example, if the phenotype established from a LRRK2 mutant form of Parkinson's Disease and the phenotype from other patients' cells with more gene variants are the same, they could be called similar and could be treated with the same candidate agent.

As used herein, a “genetic disease” refers to any pathological condition that is directly or indirectly correlated to at least one genetic mutation. As used herein, the phrase “mutation” refers to a specific genetic change in the nucleotide sequence of the sample in comparison to the genetic sequence at the same position or location in the wild-type sample. Exemplary genetic diseases include, but are not limited to, 1p36 deletion syndrome; 18p deletion syndrome; 21-hydroxylase deficiency; Alpha 1-antitrypsin deficiency; AAA syndrome (achalasia-addisonianism-alacrima); Aarskog-Scott syndrome; ABCD syndrome; Aceruloplasminemia; Acheiropodia; Achondrogenesis type II; achondroplasia FGFR3; Acute intermittent porphyria; adenylosuccinate lyase deficiency; Adrenoleukodystrophy; Alagille syndrome; ADULT syndrome; Aicardi-Goutières syndrome; Albinism; Alexander disease; alkaptonuria; Alport syndrome; Alternating hemiplegia of childhood; Amyotrophic lateral sclerosis; Alström syndrome; Alzheimer's disease; Amelogenesis imperfect; Aminolevulinic acid dehydratase deficiency porphyria; Androgen insensitivity syndrome; Angelman syndrome; Apert syndrome; Arthrogryposis-renal dysfunction-cholestasis syndrome; Ataxia telangiectasia; Axenfeld syndrome; Beare-Stevenson cutis gyrata syndrome; Beckwith-Wiedemann syndrome; Benjamin syndrome; biotinidase deficiency; Björnstad syndrome; Bloom syndrome; Birt-Hogg-Dube syndrome; Brody myopathy; Brunner syndrome; CADASIL syndrome; CARASIL syndrome; Chronic granulomatous disorder; Campomelic dysplasia X; Canavan disease; Carpenter Syndrome; Cerebral dysgenesis-neuropathy-ichthyosis-keratoderma syndrome (SEDNIK); Cystic fibrosis CFTR (7q31.2); Charcot-Marie-Tooth disease; CHARGE syndrome; Chédiak-Higashi syndrome; Cleidocranial dysostosis; Cockayne syndrome; Coffin-Lowry syndrome; Cohen syndrome; collagenopathy, types II and XI; Congenital insensitivity to pain with anhidrosis (CIPA); Cowden syndrome; CPO deficiency (coproporphyria); Cranio-lenticulo-sutural dysplasia; Cri du chat; Crohn's disease; Crouzon syndrome; Crouzonodermoskeletal syndrome (Crouzon syndrome with acanthosis nigricans; Darier's disease; Dent's disease (Genetic hypercalciuria); Denys-Drash syndrome; De Grouchy syndrome; Di George's syndrome; Distal hereditary motor neuropathies, multiple types; Edwards Syndrome; Ehlers-Danlos syndrome; Emery-Dreifuss syndrome; Erythropoietic protoporphyria; Fanconi anemia (FA); Fabry disease; factor V Leiden thrombophilia; familial adenomatous polyposis; familial dysautonomia; Feingold syndrome; FG syndrome; Friedreich's ataxia; G6PD deficiency; galactosemia; Gaucher disease GBA (1); Gillespie syndrome; Glutaric aciduria, type I and type 2; GRACILE syndrome; Griscelli syndrome; Hailey-Hailey disease; Harlequin type ichthyosis; Hemochromatosis; hemophilia FVIII; Hepatoerythropoietic porphyria; Hereditary coproporphyria; Hereditary hemorrhagic telangiectasia (Osler-Weber-Rendu syndrome); Hereditary Inclusion Body Myopathy; Hereditary multiple exostoses; Hereditary spastic paraplegia (infantile-onset ascending hereditary spastic paralysis); Hermansky-Pudlak syndrome; Hereditary neuropathy with liability to pressure palsies (HNPP); Heterotaxy; homocystinuria CBS (gene); Huntington's disease; Hunter syndrome; Hurler syndrome; Hutchinson-Gilford progeria syndrome; Hyperlysinemia; hyperoxaluria; hyperphenylalaninemia; Hypoalphalipoproteinemia (Tangier disease); Hypochondrogenesis; Hypochondroplasia; Immunodeficiency, centromere instability and facial anomalies syndrome (ICF syndrome); Incontinentia pigmenti; Ischiopatellar dysplasia; Isodicentric; Jackson-Weiss syndrome; Joubert syndrome; Juvenile Primary Lateral Sclerosis (JPLS); Keloid disorder; Kniest dysplasia; Kosaki overgrowth syndrome; Krabbe disease; Kufor-Rakeb syndrome; LCAT deficiency; Lesch-Nyhan syndrome; Li-Fraumeni syndrome; Lynch Syndrome; lipoprotein lipase deficiency recessive; Malignant hyperthermia; Maple syrup urine disease; Marfan syndrome; Maroteaux-Lamy syndrome; McCune-Albright syndrome; McLeod syndrome; MEDNIK syndrome; Mediterranean fever, familial, Menkes disease; Methemoglobinemia; methylmalonic academia; Micro syndrome; Microcephaly; Morquio syndrome; Mowat-Wilson syndrome; Muenke syndrome; Multiple endocrine neoplasia type 1 (Wermer's syndrome); Multiple endocrine neoplasia type 2; Muscular dystrophy; Muscular dystrophy, Duchenne and Becker type; Myostatin-related muscle hypertrophy; myotonic dystrophy; Natowicz syndrome; Neurofibromatosis type I; Neurofibromatosis type II; Niemann-Pick disease; Nonketotic hyperglycinemia; Nonsyndromic deafness; Noonan syndrome; Norman-Roberts syndrome; Ogden syndrome; Omenn syndrome; osteogenesis imperfect; Pantothenate kinase-associated neurodegeneration; Patau Syndrome (Trisomy 13); PCC deficiency (propionic acidemia); Porphyria cutanea tarda (PCT); Pendred syndrome PDS (7); Peutz-Jeghers syndrome; Pfeiffer syndrome; phenylketonuria; Pipecolic academia; Pitt-Hopkins syndrome; Polycystic kidney disease PKD1 (16) or PKD2 (4); Polycystic Ovarian Syndrome (PCOS); porphyria; Prader-Willi syndrome; Primary ciliary dyskinesia (PCD); primary pulmonary hypertension; protein C deficiency; protein S deficiency; Pseudo-Gaucher disease; Pseudoxanthoma elasticum; Retinitis pigmentosa; Rett syndrome; Roberts syndrome; Rubinstein-Taybi syndrome (RSTS); Sandhoff disease; Sanfilippo syndrome; Schwartz-Jampel syndrome; spondyloepiphyseal dysplasia congenita (SED); Shprintzen-Goldberg syndrome; sickle cell anemia; Siderius X-linked mental retardation syndrome; Sideroblastic anemia; Sly syndrome; Smith-Lemli-Opitz syndrome; Smith Magenis Syndrome; Spinal muscular atrophy; Spinocerebellar ataxia (types 1-29); SSB syndrome (SADDAN); Stargardt disease (macular degeneration); Stickler syndrome (multiple forms); Strudwick syndrome (spondyloepimetaphyseal dysplasia, Strudwick type); Tay-Sachs disease; Tetrahydrobiopterin deficiency; Thanatophoric dysplasia; Treacher Collins syndrome; Tuberous Sclerosis Complex (TSC); Turner syndrome; Usher syndrome; Variegate porphyria; von Hippel-Lindau disease; Waardenburg syndrome; Weissenbacher-Zweymüller syndrome; Williams syndrome; Wilson disease; Woodhouse-Sakati syndrome; Wolf-Hirschhorn syndrome; Xeroderma pigmentosum; X-linked mental retardation and macroorchidism (fragile X syndrome); X-linked spinal-bulbar muscle atrophy (spinal and bulbar muscular atrophy); Xp11.22 deletion; X-linked severe combined immunodeficiency (X-SCID); X-linked sideroblastic anemia (XLSA); 47,XXX (triple X syndrome); XXXX syndrome (48, XXXX); XXXXX syndrome (49, XXXXX); XYY syndrome (47,XYY); and Zellweger syndrome. Genetic mutations that result in a genetic disease described above are known in the art and can be identified by a skilled clinician.

Next, within each phenotype cluster for a given disease, the inventors have developed processes and algorithms (e.g., unsupervised clustering algorithms) to identify convergent genetic mutations within each phenotype cluster that are potential druggable targets for that phenotype cluster for the disease. Then, the inventor(s) have differentiated stem cells (e.g., iPSCs) from the patients within each phenotype cluster, for example to develop organoids or other tissues. “Differentiation” refers to the process whereby pluripotent stem cells (e.g., iPSCs) acquire gene expression profiles, markers, and/or structural and functional characteristics known to be associated with mature cells, which are more specialized, closer to becoming terminally differentiated cells, and/or are incapable of further division or differentiation. In many cases, the cells are differentiated into all three germ lines and a variety of target tissues based on, for example, the observed phenotypes from the EHR records. These differentiated cells are then tested for their phenotypes to identify the expressed defects in the various tissues types or organoids.

As used herein, “induced pluripotent stem cells” or “iPSC” refer to pluripotent stem cells obtained from a non-pluripotent cell, typically an adult somatic cell (a cell of the body, rather than gametes or an embryo), by inducing a “forced” expression of certain genes. iPSCs are believed to be similar to natural pluripotent stem cells, such as embryo stem cells (ESCs) in many respects. iPSCs are not adult stem cells, but reprogrammed cells given pluripotent capabilities. iPSCs do not refer to cells as they are found in nature. iPSCs can be obtained using common methods known in the art, e.g., as described in Takahashi et al. Cell 2007; 131: 861-872; U.S. Pat. Nos. 8,802,438, 9,018,010, 8,691,574, 9,068,170, 8,852,941, 8,871,504; and US Published Application Nos: US20120021519, US20080038820, UA20110263015, US20030161817, the contents of which are incorporated herein by reference in their entireties.

Additionally, the differentiated cells can then be tested with candidate agents (for instance based on the druggable targets identified by the common genetic pathways) to determine whether the agents reverse the defective phenotypes identified in the differentiated tissues or organoids. If any of the candidate agents reverse the phenotypes, they are good candidates for treatments for the phenotypic clusters.

Accordingly, future patients identified as belonging to the same phenotypic cluster may be treated with the successful candidate agent. This is a far superior approach than the current approach in genetic diseases (especially where the disease could be impacted by numerous mutations) in treating all patients with the disease with the same drugs to treat the main symptoms or in general only reacting and treating symptoms. Thus, provided herein is a method of preventing the onset of a genetic disease. This method comprises administering to a patient having a non-common symptom for the disease or disorder (e.g., a symptom not known in the art to aid in the diagnoses of a disease or disorder, for example, a genetic disease) the candidate agent for the treatment of the disease or disorder. In one embodiment, the candidate agent prevents the onset of the disease or disorder, as observed by the occurrence of a common symptom the disease or disorder (e.g., a symptom known in the art to aid in the diagnoses of a disease or disorder). In one embodiment, the candidate agent treats the uncommon symptom and/or the disease or disorder (e.g., the genetic disease).

The inventor(s) have also discovered that some of these genetic mutations for a certain broad category of disease (e.g. Parkinson's) may impact and cause conditions in tissues that are seemingly unrelated to the primary presentation of the disease (e.g. outside the nervous tissue). The inventor(s) discovered that these extraneous tissue phenotypes may be specific and variable to the phenotype clusters (sub-classes of the disease). Accordingly, certain genetic mutations that may be expressed in nervous tissue, may also cause, for instance, lung issues or gastrointestinal issues. This is because the same mutations responsible for the main classic phenotypes in the brain tissue, for example, are also present in other tissues.

Accordingly, the inventor(s) have discovered that this phenotypic-genetic classification allows them to treat genetic druggable targets that may prevent or treat issues unrelated to the classic symptoms—that were previously unknown to be associated with the disease. Thus, this approach is vastly superior to the current approach of treating common, expressed symptoms, and has unlocked the potential to identify and personalize treatments for patients based on their sub-classes of complex genetic and environmentally caused diseases.

### Early Identification of Disease Sub-Class Based on Electronic Health Records and Genetic Data

Finally, this approach has also allowed caregivers and providers to develop algorithms integrated with EHR systems that identify patients with early (and previously unknown) symptoms associated with a sub-class of a complex genetic disease. For instance, if a patient has early lung symptoms of Parkinson's and a diagnosis code is issued related to the lung symptoms, if a server and database maintained genetic data of that patient, an algorithm could continually be run to identify and flag patients that may have the early stages of Parkinson's that were previously unknown.

For instance, the genetic data could be maintained in a database for each patient, and the patient's diagnosis codes could be stored. As each new diagnosis code for a particular patient was received, the algorithm(s) could be run to determine whether the patient should be flagged for any potential for predicting the onset of a future disease, for instance. This will allow an automated system to be developed that would: (1) automatically flag patients that may have a phenotypic-genetic sub-class of a disease, (2) identify potential treatments for the patient that were discovered in the above drug discovery platform, and (3) treating one symptom and preventing future ones through a common druggable pathway discovered for the phenotype-genetic cluster.

### Systems and Data Architecture

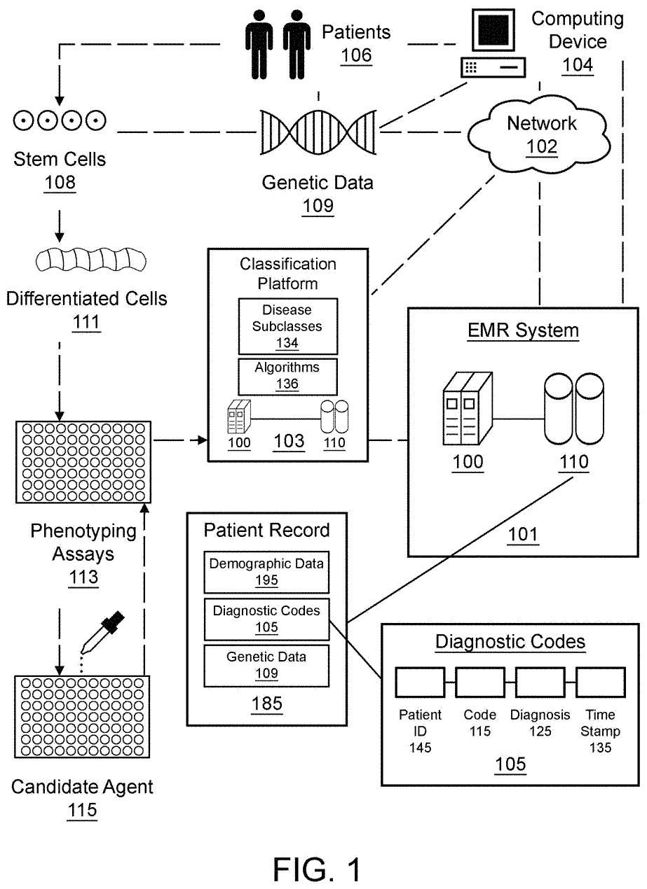

FIG. 1 is an overview of an example of a system that may be utilized to implement the platform described herein. For instance, in some examples the system may include an electronic medical records or electronic health records (“EHR”) system 101. The EHR system 101 may include computing device 100, and a database 110 to store the records, algorithms and other related data. In some embodiments, computing device 100 may be a server. In some embodiments, computing device 100 may include multiple distributed computing devices (e.g., multiple servers). The database 110 may be a relational database or any other suitable type of database, as aspects of the technology described herein are not limited in this respect.

In some embodiments, the EHR system 101 may store multiple electronic health records. An electronic health record for a patient (a “patient record”) may be stored using one or multiple data structures in database 110. An patient record may include any of numerous types of information about a patient. For example, patient record 185 may include demographic data 195, diagnostic codes 105, and genetic data 109. In some embodiments, diagnostic codes 105 may include data fields stored in a database 110 that includes a patient ID 145 (e.g., patient name, identifier, or other information), an alphanumeric code 115 (for instance a code from the ICD-9 codes, or International Classification of diseases or other similar coding systems), and/or a time stamp 135. In some embodiments, the time stamp 135 may include the date/time of diagnosis or other relevant timing information including the age of the patient.

In some embodiments, genetic data 109 may include sequencing data obtained from a biological sample of the patient. For example, the sequencing data may include DNA sequencing data, RNA sequencing data, and/or proteome sequencing data. In some examples, genetic data 109 or other data types maybe stored in a separate database 110. Genetic data 109 may be imported as a text delimited file or other suitable file that includes data fields that may include a list of mutations at certain locations, for instance. In other embodiments, the genetic data 109 may only include mutations that are relevant for the current algorithms that have been developed to classify diseases into sub-categories. In some embodiments, genetic data 109 could be a copy of the whole genome sequenced from an individual patient.

In some embodiments, a patient record may include speech and/or text data derived from notes dictated and/or types by one or more clinicians interacting with the patient. The text data may have entered as text by a clinician and/or may have been obtained by using automated speech recognition on speech dictated by the clinician. At least some of the text in a patient record may be unstructured natural language text.

The system may also include a classification platform 103 that includes a server 100 and database 110. The classification platform 103 may communicate with the EHR system 101 through an API or other communication protocol. In some examples, the classification platform 103 may extract data from the EMR system 101 in order to analyze patient records 185 to identify classifications of diseases based on patient data. In some examples, the classification platform 103 may include data relating to disease sub-classes 134 and algorithms 136 that run on its server 100. In other examples, the sub-classification platform 103 may be stored on the EMR system 101 and the algorithms 136 and disease sub-classes 134 would be stored in the database 110 of the EMR system 101.

In some embodiments, the classification platform 103 may cluster patients based on certain information stored as part of and/or referenced by patient records 185. For example, the classification platform 103 may cluster patients based at least in part on the diagnostic codes in patient records. Additionally or alternatively, the classification platform 13 may cluster patients based on featured derived from text (e.g., unstructured text) obtained from the patient's doctor(s). Such features may be derived using natural language processing techniques. For example, in some embodiments, such feature may be derived using the Apache CTAKES system for extracting clinical information from electronic health record unstructured text.

The classification platform 103 may output classifications or groups of patients that belong to a disease classification 134 including its patient record 185 to a computing device 104. Accordingly, in some examples, a caregiver, provider, or researcher, will then take that information and harvest stem cells 108 from the patient(s) 106 (e.g. iPS cells, which are generated from adult, differentiated cells obtained from the patient, e.g., using protocols described herein) and differentiate them into mature, differentiated cells 111. In some examples, the stem cells 108 may be reprogrammed into iPS cells, or other types of stem cells may be harvested, for example, embryonic stem cells, non-embryonic (adult) stem cells, or cord blood stem cells. In other examples, they may be stem cells 108 having mutations that match convergent mutations found in patients 106.

The differentiated cells 111 may be differentiated into all types of tissues from all germ layers according to known protocols, for instance as described in Hoffman, et. al, “New considerations for hiPSC-based models of neuropsychiatric disorders,” Mol. Psy. (2018); Quadrato and Arlotta, “Present and future of modeling human brain development in 3D organoids,” Curr. Opin. Cell Bio. 49:47-52 (2017); and Falk, et al, “Modeling psychiatric disorders: from genomic findings to cellular phenotypes,” Mol. Psychiatry 21(9):1167-73 (2016), U.S. Pat. Nos. 8,349,609, 9,123,120, 9,376,664, 9,487,751, 9,732,319, 8,372,642, 10,024,870, 9,938,503, 6,574,179; and US Published Application Nos. US20170009210, US20130029416, US20180072992, US20160222348, US20170027994, the content of which are incorporated herein in their entirety. Exemplary tissues include, but are not limited to, neuronal tissue, nervous tissue, cardiac tissue, lung tissue, hepatic tissue, splenic tissue, smooth muscle, skeletal muscle, colon tissue, intestinal tissue, gut tissue, epidermis tissue, retina tissue, blood cells, kidney tissue, bone, and connective tissue. In other examples, differentiated cells 111 may be differentiated into organoids according to known methods, for instance as described in U.S. Pat. Nos. 9,765,301, 9,725,124, 10,087,417, 9,856,458, 9,771,562, 9,375,514, 5,627,021; US Published Application Nos. US20140017140, US20170292116, US20170267977, US20170205396; and International Published Application Nos. WO2008004598, WO2013063588, WO2017142069, WO2018047914, WO2017160671, WO2016094948, WO2018124450, the content of which are incorporated herein in their entirety. Exemplary types of organoids include, but are not limited to, cerebral, intestinal, stomach/gastric, lingual, thyroid, thymic, testicular, hepatic, pancreatic, epithelial, lung, kidney, gastruloid, and cardiac.

The differentiated cells 111 may be subjected to different types of assays 113 to identify the phenotype of the differentiated cells and identify any abnormal or disease phenotypes. A variety of assays 113 may be utilized, which includes, but is not limited to: (1) image-based assays examining cell properties such as structural, proliferation, differentiation and migration, cell death markers or oxidative stress dyes, (2) RNA sequencing (bulk or single cell), (3) PCR-based assays, (4) gene profiling, (5) electrophysiology testing, and other assays. The results may indicate one or more maladies, abnormalities, or other defects that indicate the mechanism of disease in a patient.

In some examples, the system will also include different candidate agents 115 that are applied to the differentiated cells 111. Agents 115 may include but are not limited to, pharmaceutical compounds, drugs, biologics, small molecules, antibodies or antibody reagents, NANOBODIES®, peptides, genome editing systems (e.g., CRISPR, TALEN, and meganucleases), antisense oligonucleotides, RNA interference molecules (e.g., small interfering RNAs (siRNAs), micro RNAs (miRNAs), or short hairpin RNAs (shRNAs)), and genetically modified or engineered cells (e.g., CAR T cells). Different methods of introducing the agents may be utilized and should be based on the type or nature of the agent. Additionally, the differentiated cells 111 may be subsequently assayed to determine whether the agents 115 reversed the abnormal phenotype detected by the assays 113.

In other examples, a variety of other hardware, computing devices, network architecture, and other arrangements could be utilized.

### Methods—Drug Discovery

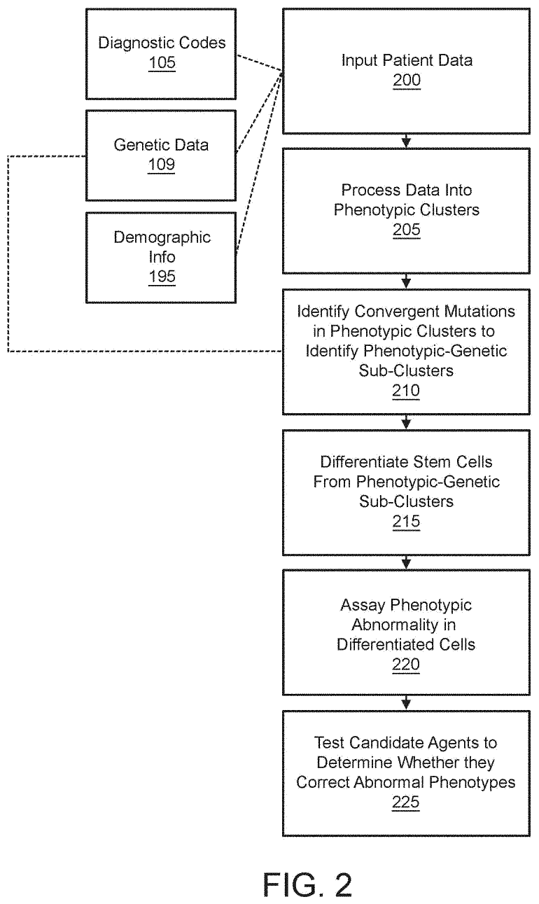

In some examples, the system could be utilized as a drug discovery platform or mechanistic discovery platform that determines the mechanism and treatments for sub-classifications of complex genetic diseases, including neural related diseases. FIG. 2 illustrates an example of one embodiment of a method implemented with the system of FIG. 1, for example, that may identify phenotypic-genetic sub-classifications of diseases 134, and then identify potential treatments for those sub-classifications. In other examples, CRISPR related implementation could be utilized to identify or confirm genetic related mechanisms underlying phenotypes.

First, the system may include a processor or computing device 104 that inputs patient data 200, that may include a variety of patient records 185 with diagnostic codes 105, genetic data 109 and demographic information 195 for each of the patients 106. In some examples, this may include first querying an EMR system database 110 to identify all patient records 185 that have a particular diagnosis of interest, for example, Parkinson's disease by identifying the correct code 115 linked to the patient ID 145.

Next, those patients may be filtered for various confounding variables. This will output an initial pool of patients all with a diagnosis of interest. After that and in some examples, all of the diagnostic codes 105 for each of the patients in the pool will be gathered from the EMR system 101. In other examples, a separate database 110 with all of this information may be queried.

The system will the process the patient records 185 including diagnostic codes 105 and/or unstructured text into phenotypic clusters 205 that represent clusters or sub-classes of patients with the disease of interest (e.g. Parkinson's) that have a similar grouping of symptoms. A variety of algorithms may be utilized to generate the phenotypic clusters. For instance, the algorithm may utilize data form the phenotype-wide association study (PheWAS). In some examples, the diagnostic codes 105 may be processed into dimensional vectors representing the counts of the most common diagnostic codes in specific time windows for the patients' life timeline. In some examples, hierarchical clustering may be performed using Euclidean distance and Ward's method.

Examples of such algorithms are provided in Kohane, et al., “Comorbidity Clusters in Autism Spectrum Disorder: An Electronic Health Record Time-Series Analysis,” Ped. 133, 1 (2014), the content of which is incorporated herein in its entirety.

Next, the system may process the phenotypic clusters using an algorithm to identify convergent mutations within those clusters to output phenotypic-genetic sub clusters 210. In some embodiments, this may be the entire phenotypic cluster has convergent, identifiable mutations. In other examples, the phenotypic clusters may be sub-divided into phenotypic-genetic sub-clusters, that are sub-groups of the phenotypic clusters that have convergent mutations. The generation of phenotypic-genetic sub clusters 210 may be performed in any suitable way. For example, in some embodiments, the data in each phenotypic cluster may be further divided into phenotypic-genetic sub cluster using a clustering algorithm. The clustering algorithm may be an unsupervised clustering algorithm, non-limiting examples of which include k-means clustering, agglomerative hierarchical clustering, density-based clustering, Gaussian mixture model based clustering, and principal components analysis. Alternatively semi-supervised techniques such as auto-encoders may be employed.

Therefore, once the final phenotypic-genetic (“PhenGen”) sub-clusters have been identified, stem cells representing those PhenGen sub-clusters may be differentiated 215. For instance, iPS cells may be generated from the patients in each PhenGen sub-cluster 215, and differentiated into a desired cell type. Then, the differentiated cells may be assayed to identify phenotypic abnormalities or disease phenotypes in the differentiated cells 220. As described above, a variety of assays maybe utilized. Alternatively, the generated iPS cells may additionally be assayed to identify phenotypic abnormalities or disease phenotypes.

Then, candidate agents 115 may be applied to the differentiated cells 111 to determine whether they correct the abnormal or disease phenotype observed in the assays by repeating the assays, or performing different assays 113. In some examples, CRISPR technology may be utilized to edit key sequences in stem cells to mimic the mutations of the phenotypic-genetic sub-clusters.

If the assays 113 demonstrate the candidate agents 115 correct, partial correct, or improve the disease or abnormal phenotype (for instance, relative to control, non-disease phenotype iPS cells which are differentiated with the same protocol), they will be saved as potential candidate for treatment of those phenotypic-genetic sub-clusters. In some examples, long term studies may be done to determine whether the candidate agents 115 not only reverse abnormal phenotypes that were observed with assays, but prevent latent phenotypes with a known timeline for expression. Accordingly, some candidate agents 115 may be a method of treatment of a phenotype in a phenotypic-genetic sub-cluster, and/or may be preventive of phenotypes that will be expressed down the line, for instance (in the case of Parkinson's) in neural or non-neural tissues. As many of the mutations responsible for the diseased phenotype may be expressed in multiple tissue types, it is possible some of the candidate agents 115 identified may reverse or prevent multiple maladies associated with a disease that may be expressed in different tissues.

### Methods—Early Detection of Diseases

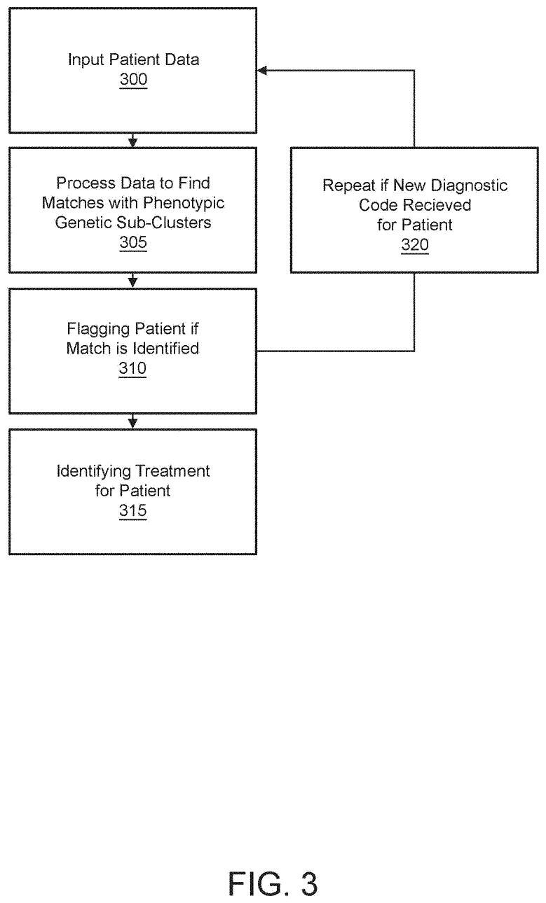

In some examples, after disease phenotype-genetic sub-clusters are identified and associated treatments discovered, these systems and methods may be implemented to monitor provider's EHR records to identify patients that may fit into the phenotypic-genetic sub-cluster and flag them for treatments. For instance, FIG. 3 illustrates an example software process that may integrate with a provider's medical records to continually monitor patients for the early signs of complex genetic disease.

First, the system may input patient data 300 associated with a patient record 185, that includes at least diagnostic codes 105 and genetic data 109. In some examples, the patient data 300 may include time stamps 135 on the diagnostic codes 105.

Next, the system may utilize matching algorithms 136 to determine whether the patient record 185 is a match for any of the phenotypic-genetic sub-clusters 305. This may determine the probability for which this patient likely fits without the phenotypic genetic sub-cluster 305 of a certain disease, without necessarily having a diagnostic code 105 associated with the disease. For instance, the system may determine based on the patient's genetic data 109, non-classic diagnostic codes, and/or unstructured text that a patient is likely to develop Parkinson's disease within a few years.

Then, the system may flag the patient if a match within a certain threshold is determined 310. This may include outputting an indication of the likely timeline for the development of certain phenotypes of the disease, for instance classic neurological symptoms in the case of Parkinson's disease. Additionally, the system may indicate any potential treatments associated with that phenotypic-genetic sub-cluster of the disease as a potential treatment for the patient. In some instances, notifications will automatically sent to a computing device 109 of a provider or caregiver to allow them to proactively reach out to the patient 106 and recommend treatment.

### REFERENCES

1. Bastarache L et al., 2018. Phenotype risk scores identify patients with unrecognized Mendelian disease patterns. Science 359:1233-39.
2. Bock C et al., 2011. Reference Maps of human ES and iPS cell variation enable high-throughput characterization of pluripotent cell lines. Cell 144:439-52. PMCID: PMC3063454.
3. Denny J C et al., 2013. Systematic comparison of phenome-wide association study of electronic medical record data and genome-wide association study data. Nature Biotechnol 31:1102-100. PMCID: PMC3969265.
4. Fuji R N et al., 2015. Effect of selective LRRK2 kinase inhibition on nonhuman primate lung. Sci Transl Med 7:273ra15.
5. Hoffmann et al., 2017. Genome-wide association analyses using electronic health records identify new loci influencing blood pressure variation. Nature Genet 49:54-64. PMCID: PMC4364461.
6. Hui K Y et al., 2018. Functional variants in the LRRK2 gene confer shared effects on risk for Crohn's disease and Parkinson's disease. Sci Transl Med 10:eaai7795.
7. Kohane I S et al., 2012. The co-morbidity burden of children and young adults with autism spectrum disorders. PloS One 7:e33224. PMCID: PMC3325235.
8. Lambert J C et al., 2013. Meta-analysis of 74,046 individuals identifies 11 new susceptibility loci for Alzheimer's disease. Nature Genet 45:1452-8. PMCID: PMC389625.
9. Liu et al., 2012. Progressive degeneration of human neural stem cells caused by pathogenic LRRK2. Nature 491:603-7. PMCID: PMC3504651.
10. Lonsdale, J et al., 2013. The genotype-tissue expression (GTEx) project. Nature Genet 45:580-5.
11. McCauley K B et al., 2017. Efficient Derivation of Functional Human Airway Epithelium from Pluripotent Stem Cells via Temporal Regulation of Wnt Signaling. Cell Stem Cell 20:844-57.
12. Nalls M A et al., 2014. Large-scale meta-analysis of genome-wide association data identifies six new risk loci for Parkinson's disease. Nature Genet 46:989-93. PMCID: PMC4146673.
13. Ng S-Y et al., 2015. Genome-wide RNA-Seq of Human Motor Neurons Implicates Selective ER Stress Activation in Spinal Muscular Atrophy. Cell Stem Cell 17:569-84. PMCID: PMC4839185.
Ordureau A et al., 2018. Dynamics of PARKIN-Dependent Mitochondrial Ubiquitylation in Induced Neurons and Model Systems Revealed by Digital Snapshot Proteomics. Mol Cell 70:211-27. PMCID: PMC5910199.
14. Paik E J et al., 2017. Using Intracellular Markers to Identify a Novel Set of Surface Markers for Live Cell Purification from a Heterogeneous hIPSC Culture. Sci Rep 8:804. PMCID: PMC5770419.Pardiñas A F et al., 2018. Common schizophrenia alleles are enriched in mutation-intolerant genes and in regions under strong background selection. Nature Genet 50:381-89. PMCID: PMC5918692.
15. Rodriguez-Muela N et al., 2017. Single Cell Analysis of SMN Reveals Its Broader Role in Neuromuscular Disease. Cell Reports 18:1484-98. PMCID: PMC5463539.
16. Ross O A, et al., 2011. Association of LRRK2 exonic variants with susceptibility to Parkinson's disease: a case-control study. The Lancet Neurol 10:898-908. PMCID: PMC3208320.
17. Takahashi Y et al., 2018. A Refined Culture System for Human Induced Pluripotent Stem Cell-Derived Intestinal Epithelial Organoids. Stem Cell Reports 10:314-28. PMCID: PMC5768885.

### EXAMPLES

The following examples are provided to better illustrate the claimed invention and are not intended to be interpreted as limiting the scope of the invention. To the extent that specific materials or steps are mentioned, it is merely for purposes of illustration and is not intended to limit the invention. One skilled in the art may develop equivalent means or reactants without the exercise of inventive capacity and without departing from the scope of the invention.

### Example 1: Parkinson's Disease

Following, is one example of how the above disclosure can be utilized to sub-classify forms Parkinson's disease and identify and develop treatments for them. In this example, the platform utilizes LRRK2, a well-known PD-associated gene, as a starting point for testing the value of EHR and iPSCs in achieving a more comprehensive understanding of complex diseases. For instance, the subjects can be first classified with EHR data, and then their genetic information can sub-classify them. This Examples utilizes EHR data from Partners Healthcare, a health care provider with extensive patient data and medical records. Particularly, comprehensive health information covering complete disease pathogenesis including prodromal state, initial diagnosis, and progression is captured within EHR data at Partners. The overall Partners EHR database includes over 6 million individuals, with associated genomic data (Illumina SNP chips) on over 20,000 of them. Additionally, the HMS Biomarker Core has whole genome sequences and/or specific coverage of PD-associated genes from 800+ individuals that are also captured within the Partners EHR. For this project, variants in LRRK2, a gene with many PD-associated variants detailed in the ClinVar database, the most studied being G2019S, affect health. While LRRK2 is associated with PD, there may be other phenotypes associated with the gene (even in individuals not yet diagnosed with PD) that will reveal more about the gene's role in other tissues, as mentioned above.

An initial exploration of subjects with genomic data captured by the Partners Biobank revealed a total of 43 subjects with the top pathogenic LRRK2 variants and over 5000 with common variants identified through GWAS studies (Ross et al., 2011; Table 1). Data from heterozygotes for GWAS variants can be explored independently from the pathogenic variants. Controls are those lacking any known LRRK2 protein coding variants. It is important to reiterate that the analysis would include patients not (yet) diagnosed with PD, as the goal here is to understand how PD-associated gene variants affect total body health. While some of these patients may eventually develop PD, their data will still be valuable in understanding the role of each gene in human health.

TABLE 1
Patients with LRRK2 Variants in Partners Biobank

| Protein change | rs# | Het | Hom | Pathogenic |
|---|---|---|---|---|
| G2019S | rs34637584 | 40 | 0 | Yes |
| R1441C | rs33939927 | 3 | 0 | Yes |
| N1437H | rs74163686 | 0 | 0 | Yes |
| R1441H | rs34995376 | 0 | 0 | Yes |
| Y1699C | rs35801418 | 0 | 0 | Yes |
| I2020T | rs34015634 | 0 | 0 | Yes |
| L953L | rs7966550 | 4075 | 279 | No (GWAS) |
| R1398H | rs7133914 | 2956 | 174 | No (GWAS) |
| N551K | rs7308720 | 2901 | 156 | No (GWAS) |
| N2081D | rs33995883 | 817 | 14 | No (GWAS) |
| A419V | rs34594498 | 7 | 0 | No (GWAS) |

The system could be applied to identify differential diagnoses occurring at any time during these patients' lifetime. Data associated with clinical diagnostic codes, routinely measured laboratory values, such as lipid levels and blood pressure, and demographic information such as weight, height, etc would be used. More complex information is also available, including data captured in narrative notes, imaging studies, and other medical tests, can be utilized. A well-established technique for exploring differences in health trajectories is to group diagnoses into phenome-wide-association (PheWAS) diagnosis groups (Denny et al., 2013) that aggregate common diagnoses. The PheWAS results can be combined with data extracted from laboratory records for in vitro modeling. To determine significance of phenotypes/laboratory results between cohorts, the platform can utilize logistic regression with appropriate covariates (age, gender, coverage duration, ethnicity, etc.). When this analysis is completed, data will be compared to the results of further analysis below to see if clinical and iPSC data align at a qualitative level. An additional use for the data will include determination of which types of cells/tissues are most affected by LRRK2 variants to guide differentiation studies. For example, if subjects are shown to have altered glucose levels it may be possible to generate beta cells and test insulin secretion in response to a glucose challenge.

Once analysis is complete for LRRK2 variants, this process could be applied to examining other common PD-associated genes such as GBA and PARK2. These analyses could proceed as above. Although all of these genes are associated with greater risk of PD, their differential expression will likely be associated with different non-neural phenotypes.

Given the data with SMA discussed in Example 2 below and increasing information suggesting that non-neural tissues can be affected in neural disorders, the platform will take a broader view of the potential use of iPSCs in recapitulating disease phenotypes, focusing first on LRRK2 variants. This will be done in two ways. First, as with the SMA work described below, the platform will produce cells from all 3 germ layers using an embryoid body (EB) assay to capture potential disease proclivities more comprehensively. The hypothesis is that effects of these gene variants will not be restricted to DA neurons or even to neural lineages. To do this, control and isogenic mutant heterozygous and homozygous LRRK2^{G2019S} pluripotent cells will be harvested and grown as EBs in non-adherent wells for 14 days using standard differentiation conditions similar to those that we previously applied to SMA iPSCs (Bock et al., 2011). Five EBs per line will then be dissociated and sequenced using a 10× Genomics single cell sequencer located in the Bauer core at Harvard. These may be performed based methods established by Aviv Regev and her group at the Broad relating to single cell RNAseq analysis of cells in aging and young brains (Ximerakis et al., manuscript in preparation). The platform will be able to measure both numbers of cells generated per line for each lineage and also the aberrant expression of genes within individual cell types. Cell identity will be based on the expression of lineage specific or tissue specific markers as before. To confirm that phenotypes are related to increased LRRK2 activity, the platform will treat cells with GNE-0877, a specific LRRK2 chemical inhibitor. True mutation-associated phenotypes should be reversed with compound treatment (Liu et al., 2012). Abnormalities identified from this analysis will then be queried in the EHRs of LRRK2 mutant patients to confirm predictions made by iPSCs.

Second, once the above analysis is completed, the platform will also determine whether disease-related phenotypes that are identified from EHR data can be reproduced when starting from the pluripotent cells. For instance, the platform will use standard directed differentiation protocols to produce the cell types observed to be affected in LRRK2 mutant patients whether or not they have been diagnosed with PD. The goal will be to determine if there are alterations in the production of specific types of differentiated cells from disease vs. control lines. Based on what is observed in patient EHR, the platform can produce relevant cells using standard 2D or 3D differentiation conditions (for example: Takahashi et al, 2018; McCauley et al., 2017). Since some of the LRRK2 variants do results in PD, the platform can also produce DA neurons using methods well-established in the Rubin lab (Paik et al., 2018). The platform can determine changes in the efficiency of differentiation and also observe whether there are obvious abnormalities in cell survival. Finally, the platform may reproduce some of the known non-neural toxicities of LRRK2 inhibitors on lung cells. Together the two types of studies could help to establish how well human differentiated cells carrying disease associated gene variants can model multiple features of human disease, identification of disease subtypes, and testing of potential therapeutics.

The platform can show that observations made using iPSCs can be confirmed using EHR and that phenotypes identified by EHR can be studied using patient-derived iPSCs. Accordingly, these approaches can be applied to (a) study other forms of PD; (b) study more complex disorders including psychiatric disease; (c) group together patients who, because they are phenotypically similar in vitro (iPSC) and in vivo (EHR), might be responsive to similar treatments; (d) potentially establish algorithms to predict which non-symptomatic individuals might develop particular diseases. The ability to accomplish the proposed work leverages iPSC lines maintained and analyzed in the Rubin lab and the tens of thousands of previously sequenced patient samples at the Partners Biobank.

### Example 2: Spinal Muscular Atrophy (SMA)

Spinal muscular atrophy (SMA) is an autosomal recessive disorder, with the most prominent phenotype being neuromuscular degeneration. The genetic defect underlying SMA is a pair of missing or defective survival of motor neuron 1 (SMN1) genes that leads to an insufficient amount of SMN protein. A disease modifying paralog of SMN1, namely SMN2, also produces functional SMN protein; however, due to a point mutation it is predominantly spliced to truncated mRNA, resulting in only a fraction of the protein produced by SMN1 per copy¹. A lower copy number of SMN2 results in a more severe clinical phenotype², with copy number being closely, but not directly, associated with clinical subcategories (Types I, II, IIIa, IIIb, and IV). Determination of SMA Type is based on age of onset and best motor performance achieved³.

The neuromuscular dysfunction in SMA is attributed to a particular sensitivity of motor neurons to the low amount of SMN protein; however, there are reports using SMA mouse models and from limited human studies that multiple organ systems including the heart, vasculature, muscle, bone, lung, pancreas, liver, intestine, and testes might also be affected⁴⁻⁷. The presence of non-neuromuscular phenotypes in SMA is not entirely unexpected, as SMN1/2 genes are expressed in every tissue of the human body⁸, and are required for the viability of all eukaryotic cells⁹⁻¹¹. Moreover, SMN-increasing therapies solely targeting the central nervous system fail to reduce overall disease severity¹²⁻¹⁴ while therapies delivered peripherally rescue disease severity, possibly without entering the CNS at all¹⁵⁻¹⁶. These data combined with the recent FDA approval of Spinraza for treatment of SMA, an intrathecally delivered antisense oligonucleotide whose effects are presumably limited to cells within the spinal cord, and other therapeutics nearing clinical approval drive the need for a more complete system-wide physiological understanding of SMA¹⁷⁻¹⁸.

In this example, the platform can be utilized to determine: (1) are non-neuromuscular phenotypes found in SMA patients; and (2) which of these phenotypes occur prior to neuromuscular defects and may be directly attributable to reduced SMN levels and not a complication of muscle atrophy; and (3) can non-neuromuscular phenotypes or surrogate measures of them be used as predictors for inflection points in SMA disease severity pre-symptomatically? To investigate these important questions, the platform mined large medical datasets to develop risk-prediction models and characterize disease progression¹⁹⁻²¹.

Methods

Data was collected form a de-identified administrative database from Aetna Inc. representing 63,444,784 memberships to Aetna health insurance during a 98-month timeframe from Jan. 1, 2008 through Feb. 29, 2016. For each individual covered by a unique membership, the platform extracted gender, year of birth, enrollment duration, and unique time stamped medical records in the form of International Classification of Diseases (ICD), 9^{th} Revision, Clinical Modification codes. For each subject with ICD-10^{th} Revision codes the platform used their first ICD-10^{th} Revision code as the end of enrollment. The Harvard Medical School Institutional Review Board approved this research.

Subject/Control Selection:

Using a selection approach similar to one used to evaluate the economic burden of SMA²², the platform selected all individuals with at least 2 ICD codes for SMA. This approach is similar to one utilized to evaluate the economic burden of SMA²². Manual chart review was then performed to ensure that selected SMA patients were valid. Upon review, two issues were identified that compromised the integrity of the cohort. The first was inclusion of likely muscular dystrophy (MD) and myoneural disorder cases. To address this, inclusion required the final diagnosis to be SMA. Second, a large number of women in their late twenties and early thirties were found who represented individuals undergoing prenatal genetic testing or care for a pregnancies with a high risk of SMA. To overcome this, subjects with any pregnancy codes or codes related to pregnancy complications were excluded.

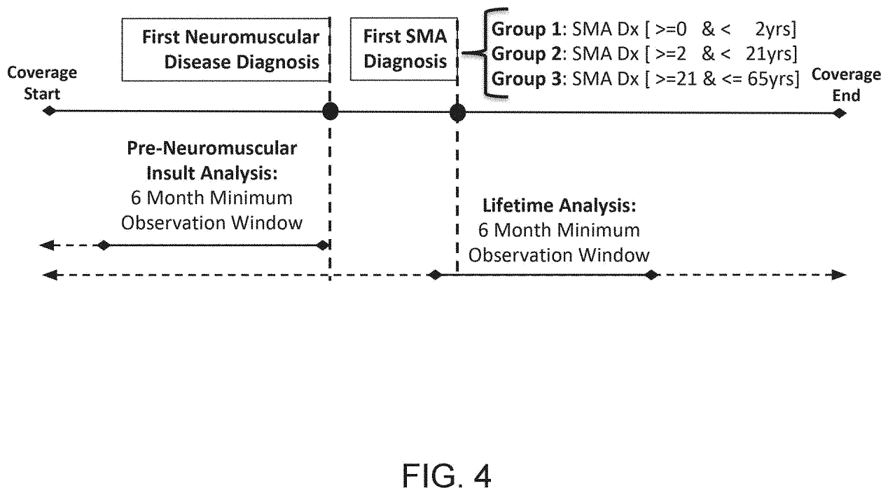

The SMA patients were then stratified into three subcategories based on age at first SMA diagnosis²³: Group 1 represented likely SMA Types I/II patients having diagnosis from birth up to 2 years of age; Group 2 represented likely SMA Types IIIa/b patients with diagnoses from 2 up to 21 years of age; and Group 3 represented likely adult-onset SMA Type IV patients with diagnoses between 21 and 65 years of age. A control population for each subcategory was selected from individuals with no SMA codes of the same age range at enrollment. Control populations for different categories may share individuals. For each subcategory, two different analyses were performed (FIG. 4). The first analysis utilized all data within the lifetime of each individual, whereas the second included, for SMA cases only, data prior to their first diagnosis of major neuromuscular disease or severe mobility, respiratory, or feeding complications. This time-point is referred to as the neuromuscular inflection point. Finally, each subject was reqyured to have at least 6 months of coverage and thus some SMA patients and control cases were excluded for pre-neuromuscular inflection point analysis.

Phenotypic Differences

To investigate the presence of differential phenotypes in SMA cases, the platform first converted ICD codes to phenome-wide-association (PheWAS) diagnosis groups²⁴. To determine significant phenotypes and covariate-adjusted odds ratio (OR), the platform regressed an indicator of the phenotype onto an indicator of SMA diagnosis using logistic regression via the glm( ) function in R-3.3.3²⁵ with gender, age at enrollment, and enrollment months as covariates. The false discovery rate (FDR) was controlled at 5% using the Benjamini-Hochberg procedure²⁶ to adjust p-values. Only phenotypes with prevalence of at least 1% in the SMA or control population were evaluated. Next, the platform took all phenotypes with adjusted p-values less than 0.05 and grouped them by physiological system to evaluate broader system wide dysfunction and repeated logistic regression analyses where now the outcome was the binary indicator of any phenotype related to the system.

Temporal Trajectories of the Onset of Categorized Phenotypes

To characterize a potential timeline of disease progression the platform computed the median time between first diagnosis associated with each physiological system and SMA diagnosis. To determine if these timelines could be attributed to a subject's enrollment duration, and not actual disease progression, the platform calculated the R² coefficient of determination between dates for all SMA cases with enrollment duration.

Results

Patient/Control Cohorts

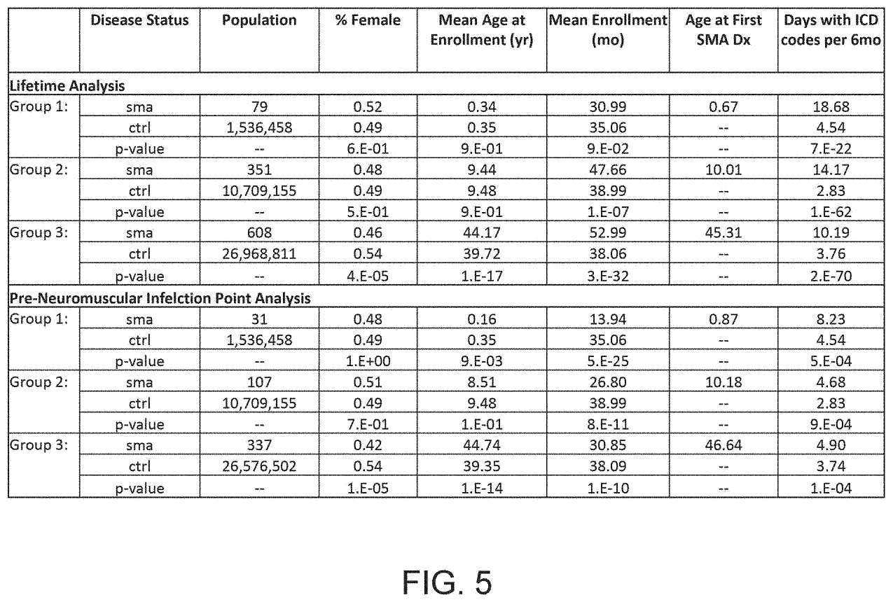

For the lifetime analysis, the platform identified 1,038 SMA cases (79 in Group 1 [0.34 yrs]; 351 in Group 2 [9.44 yrs]; and 608 in Group 3 [44.17 yrs]) and 39,214,424 controls (1,536,458 in Group 1 [0.35 yrs]; 10,709,155 in Group 2 [9.48 yrs]; and 26,968,811 in Group 3 [39.72 yrs]). For pre-neuromuscular inflection point analysis, the cohort was reduced to 475 SMA patients (31 in Group 1 [0.16 yrs]; 107 in Group 2 [8.51 yrs]; and 337 in Group 3 [44.74 yrs]) and 38,822,115 controls (1,536,458 in Group 1 [0.35 yrs]; 10,709,155 in Group 2 [9.48 yrs]; and 26,576,502 in Group 3 [39.35 yrs]). Details are presented in FIG. 5 including total population, gender, age at enrollment, mean enrollment duration, and a measure of medical utilization in the form of days with ICD codes per six-month period along with corresponding p-values. Notably, the differences in medical utilization between SMA and control cohorts are much smaller before the neuromuscular inflection point compared to over their lifetime.

Identification of the Non-Neuromuscular Phenotypes in SMA Patients

The pre-neuromuscular inflection point analyses yielded total of 17, 67, and 35 differential phenotypes in Groups 1, 2, and 3, respectively.

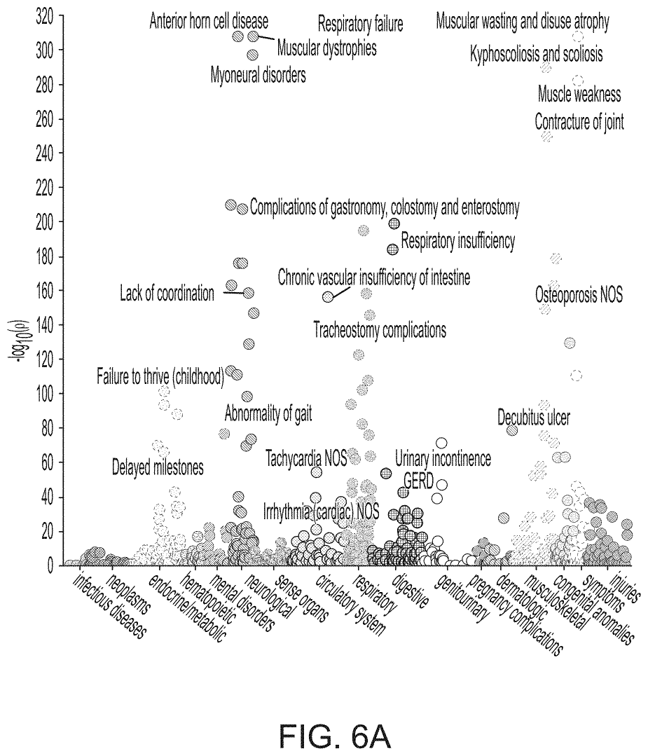

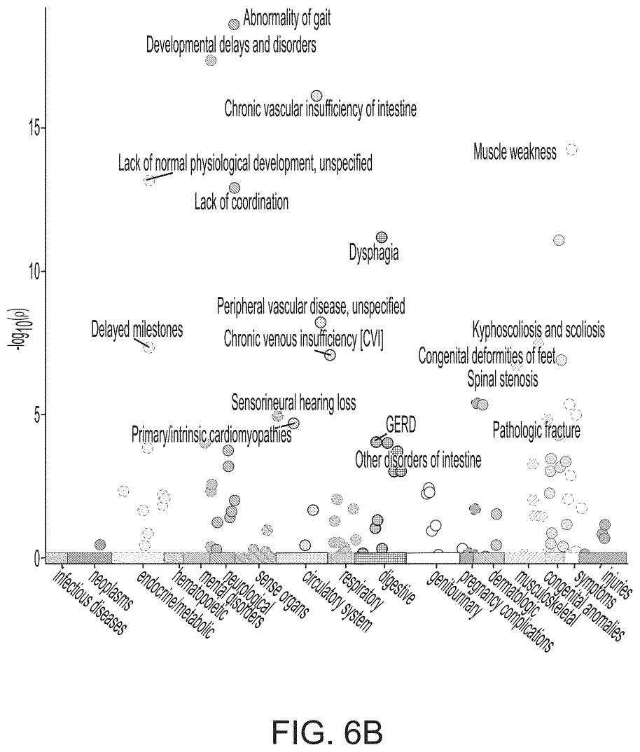

Phenotypes with significantly increased prevalence in SMA patients in Group 2 included early signs of impending neuromuscular defects such as abnormality of gait (12.1% in SMA, OR=19.1, adjusted p-value<0.001) and lack of coordination (9.3% in SMA, OR=16.1, adjusted p-value<0.001). Non-neuromuscular phenotypes with significantly increased prevalence in SMA cases included, but are not limited to: cardiovascular (cardiac shunt/heart septal defect: 4.7% in SMA, OR=5.8, adjusted p-value=0.005; peripheral vascular disease, unspecified: 1.9% in SMA, OR=122.7, adjusted p-value<0.001); gastrointestinal (GERD: 13.1% in SMA, OR=4.1, adjusted p-value<0.001; constipation: 12.1% in SMA, OR=2.8, adjusted p-value=0.01); and skeletal (kyphoscoliosis and scoliosis: 9.3% in SMA, OR=8.7, adjusted p-value<0.001; congenital deformities of feet: 6.5% in SMA, OR=11.2, adjusted p-value<0.001). Only acute pharyngitis (13.1% in SMA, OR=0.4, adjusted p-value=0.04) had lower prevalence in SMA cases. Visualization of Group 2 data is presented using a Manhattan plot in FIG. 6B alongside the lifetime analysis in FIG. 6A.

Similar to Group 2, pre-neuromuscular inflection point diagnoses from patients in Group 3 revealed early signs of neuromuscular degeneration (abnormality of gait, 3.9% of SMA, OR=2.7, adjusted p-value=0.02; spondylosis with myelopathy: 5.0% in SMA, OR=11.7, adjusted p-value<0.001; degeneration of intervertebral disc: 15.1% of SMA, OR=2.7, adjusted p-value<0.001). Non-neuromuscular phenotypes representing the following physiological systems were detected: cardiovascular (premature beats: 2.7% of SMA, OR=3.3, adjusted p-value=0.02); gastrointestinal (constipation: 7.1% of SMA, OR=2.3, adjusted p-value=0.006); skeletal (flat feet: 1.2% of SMA, OR=5.0, adjusted p-value=0.04); and sensory (disturbance of skin sensation: 9.8% of SMA, OR=2.3, adjusted p-value=0.0009). Interestingly, in milder patients who live longer and make it to reproductive age, the platform observed phenotypes in the reproductive system (testicular hypofunction: 5.3% of SMA [9.5% of males], OR=2.4, adjusted p-value=0.02; infertility, male: 1.5% of SMA [2.6% of males], OR=5.1, adjusted p-value=0.01).

Categorizing Phenotypic Differences into Physiological System

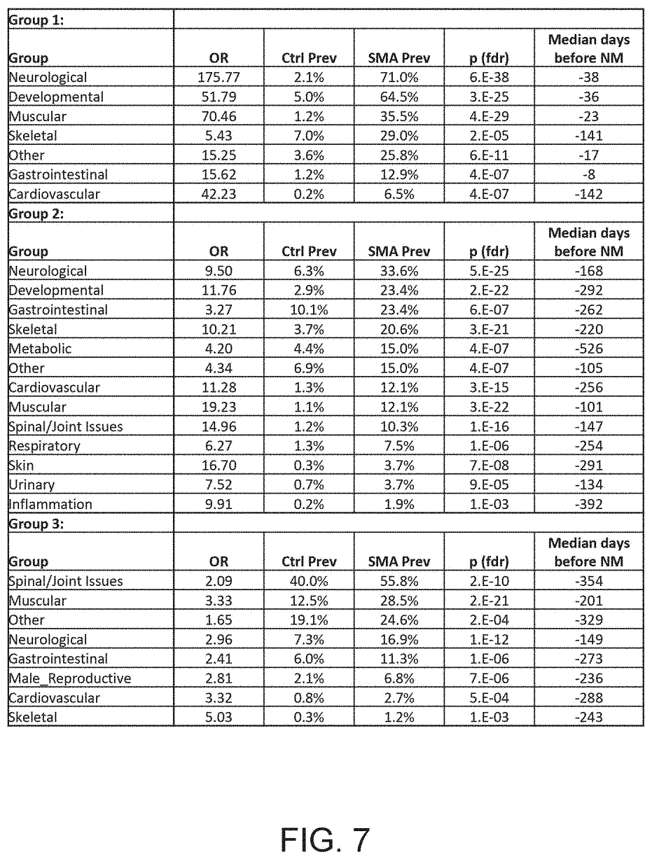

The platform then grouped phenotypes into categories representing independent physiological systems. FIG. 7 presents the odds ratio, prevalence and p-value values for each physiological system by SMA group. For Group 2, the most prevalent categorized phenotypes were neurological (33.6% in SMA, OR=9.5, adjusted p-value<0.001) and developmental (23.4% in SMA, OR=11.8, adjusted p-value=0.02). The next most prevalent phenotypes were non-neuromuscular, including gastrointestinal (23.4% in SMA, OR=3.3, adjusted p-value<0.001), skeletal (20.6% in SMA, OR=10.2, adjusted p-value<0.001), and metabolic (15.0% in SMA, OR=4.2, adjusted p-value<0.001). For Group 3, the most prevalent categorized phenotypes were diagnoses related to spinal/joint issues (55.8% in SMA, OR=2.1, adjusted p-value<0.001), muscular issues (28.5% in SMA, OR=3.3, adjusted p-value<0.001), and neurological defects (16.9% in SMA, OR=2.9, adjusted p-value<0.001). Non-neuromuscular phenotypes represented the next most prevalent in the form of gastrointestinal issues (11.3% in SMA, OR=2.4, adjusted p-value<0.001) and male reproductive issues (6.8% in SMA [11.8% in males], OR=2.8, adjusted p-value<0.001).

Temporal Trajectories of the Onset of Categorized Phenotype

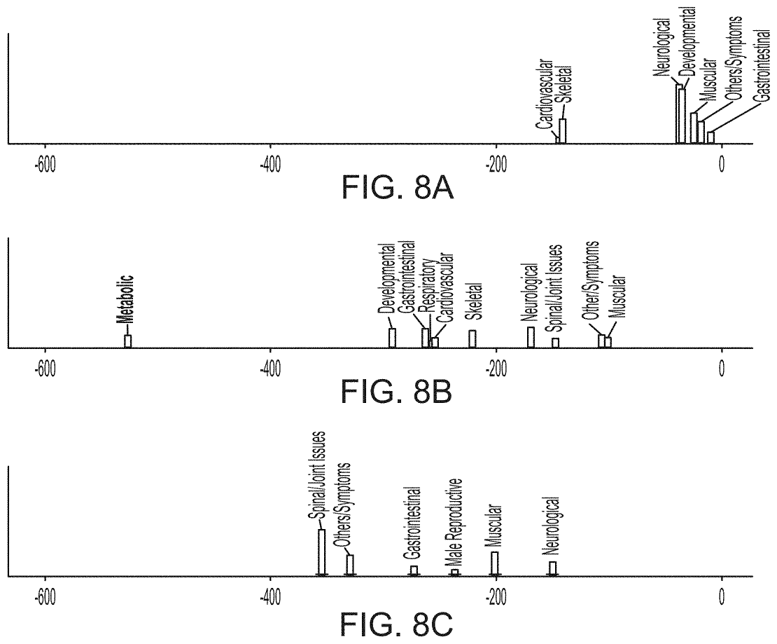

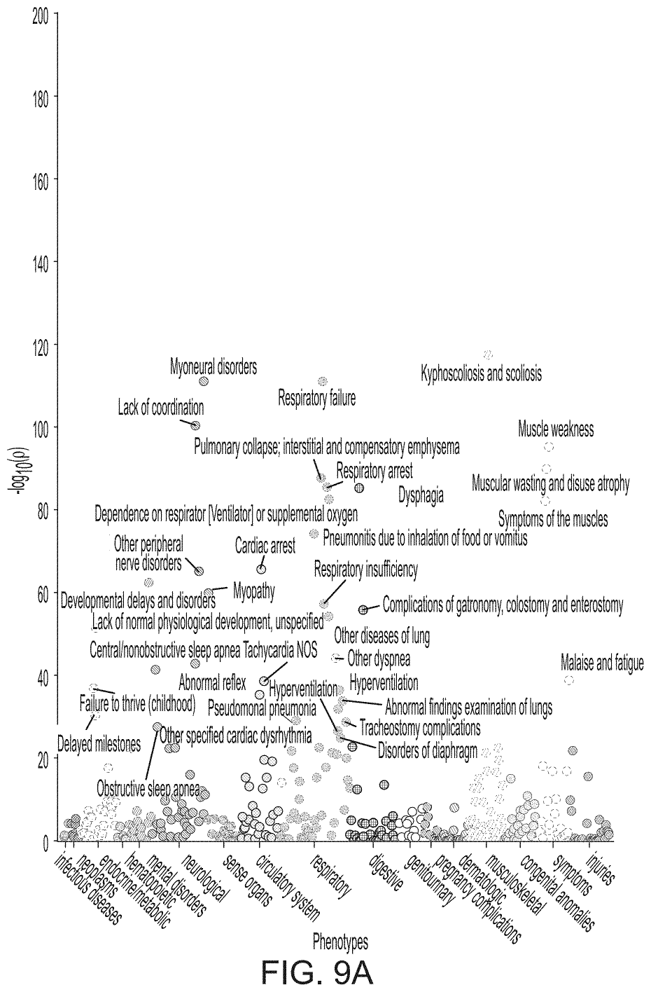

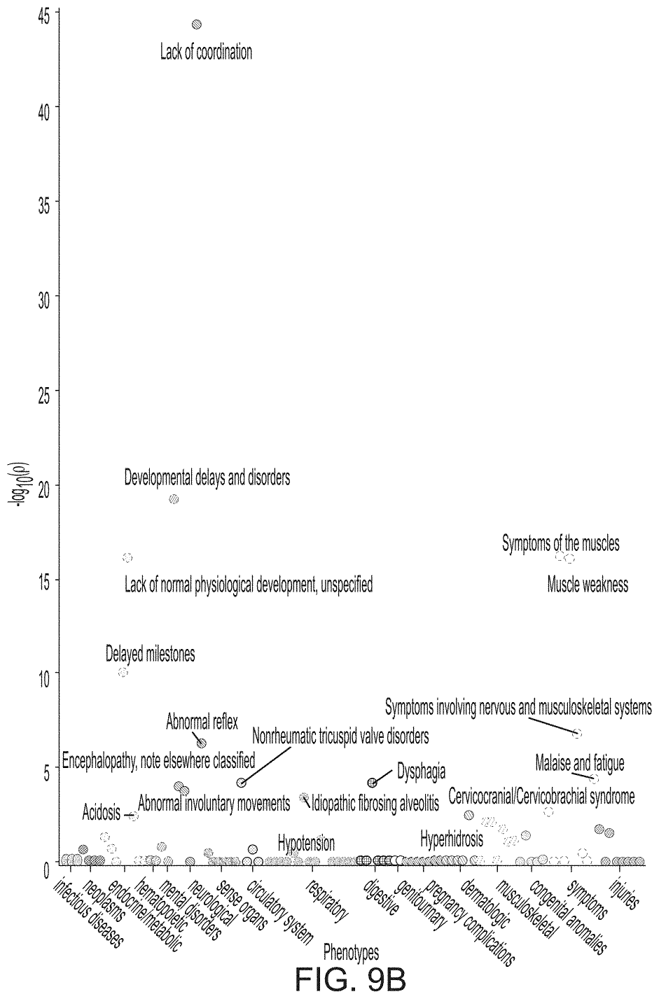

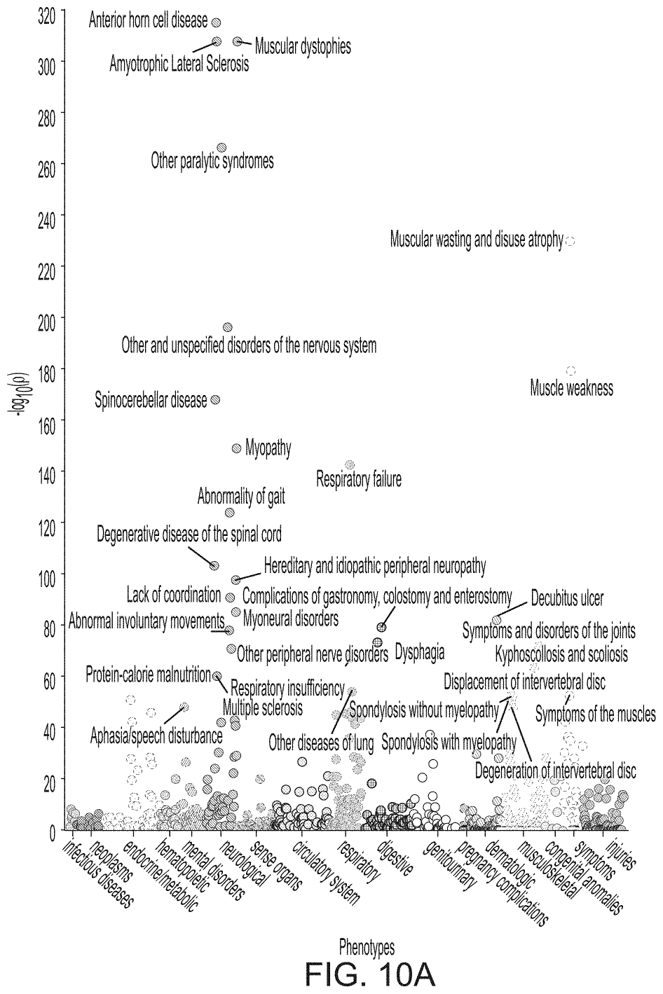

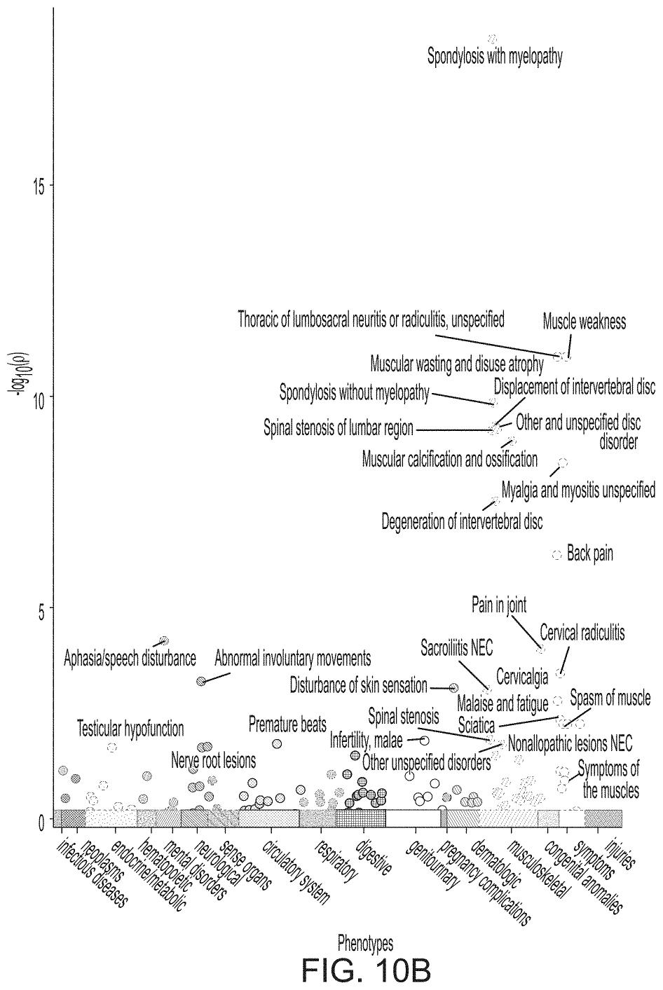

Timelines of categorized phenotypes are presented in FIG. 7 and those with at least 5% prevalence are visualized in FIG. 8. Data for Group 2, in FIG. 8B, shows that metabolic defects (−526 days) are the earliest detectable phenotypes followed by developmental (−292 days), gastrointestinal (−262 days), cardiovascular (−256 days), respiratory (−254 days), and skeletal (−220) phenotypes. Finally, patients are typically diagnosed with neurological (−168 days), spinal/joint issues (−147 days) and musculoskeletal (−101) phenotypes within the 6 months prior to their neuromuscular inflection point. FIG. 8C, shows data from Group 3 with the earliest phenotypes representing spinal/joint issues (−354 days) followed by gastrointestinal (−273 days) and male reproductive (−236 days) issues. Muscular (−201 days) and neurological (−149) phenotypes were diagnosed around 6 months prior to their neuromuscular inflection point. The R² coefficients of correlation for these results with enrollment duration were 0.21, 0.10, and 0.19 for Groups 1, 2, and 3, respectively, indicating little of the variation can be explained by enrollment duration.

Discussion

Growing evidence indicates that SMA is not solely a motor neuron disease. This includes data from a severe mouse SMA model, post-onset clinical studies or reports with small numbers of SMA patients, gene expression data showing SMN is transcribed in all tissues, and cellular data demonstrating that SMN plays a critical role in mRNA splicing. However, evidence is lacking that SMN deficiency directly translates to detectable non-neuromuscular phenotypes in humans, and, if so, at which time during disease progression. One reason for this is that historical SMA studies focus on patients after SMA onset²⁷⁻³⁰, at which point in time phenotypes can be masked by severe neuromuscular degeneration or characterized as SMA complications. Given the current and pending approvals of SMN increasing therapies, understanding the complete pathophysiology of SMA is of critical importance in order to provide more comprehensive markers of efficacy at a system-wide level and to determine whether early clinically detectable signs exists that could be used to support pre-symptomatic intervention.

In this study, for the first time, SMA patient health was investigated prior to the first signs of major neuromuscular degeneration, when the likely driver of non-neuromuscular phenotypes is likely to be SMN deficiency. The study reveals numerous non-neuromuscular phenotypes in patients with varying severities of disease, and further, demonstrates a potential ordering to their progression. Application of these findings may include: the development of early clinical diagnostic protocols that can be carried out through routine physical examination; the integration of non-traditional clinical features with existing tests to better track the health, disease progression, and therapeutic response of individual SMA patients; and the provision of a multi-systems framework that can guide the search for non-neuromuscular biomarkers that may more dynamically track patient disease before and during treatment.

These findings are encouraging in that they suggest that clinical identification of SMA patients prior to neuromuscular symptoms might be possible. Of particular interest are phenotypes seen in our pre-neuromuscular inflection point analyses that align with those reported in mouse models. For example, SMA model mice have been reported to have vascular defects that lead to necrosis of the tails and ears³¹⁻³². These results indicate that SMA patients are also more likely to be diagnosed with vascular defects, such as peripheral vascular disease, chronic venous insufficiency, and chronic vascular insufficiency of the intestines. Mouse models have been reported to present with cardiac failure, remodeling, and septal defects³³⁻³⁶, and the study found evidence of cardiomyopathies and septal defects in Group 2 patients. Additionally, mouse models of SMA have reduced numbers of intestinal villi that are blunt and club-shaped with severe intramural edema³⁶. This correlates with the findings that Groups 2 and 3 SMA patients have diagnoses of gastrointestinal disorders and dysfunction. Given the rapid turnover of intestinal cells, the structure or function of these cells may present opportunities for identification of biomarkers of therapeutic efficacy. One of the more surprising findings of the study is dysfunction in the male reproductive system. This is not unprecedented as it aligns with recent studies in mouse models of SMA showing infertility in male mice and developmental issues in their testes⁵⁻⁶ and an anecdotal human study of two subjects with atrophic testes³⁷. If male reproductive dysfunction is confirmed to be a factor in this disease, then hormone markers such as testosterone may prove to function as biomarkers of disease severity that track with therapeutic efficacy.

For each group examined in the temporal trajectory analysis, it was surprising to note that non-neuromuscular phenotypes arose prior to the presentation of any muscular or neurological symptoms. This finding is of immense clinical significance, since a key principle for SMA therapeutics is that drug interventions should act before permanent neuronal or other major cellular degeneration. Given that this study presents a first indication that non-neuromuscular symptoms may be the earliest detectable symptoms in SMA, it therefore opens the possibility for future research studies that could eventually allow for therapeutic intervention prior to the onset of any neuromuscular symptoms.

There are limitations and potential pitfalls to the study. The most obvious is that our findings are built on clinical billing data, which is susceptible to error and was not generated with the intention of being used for research purposes. Additionally, many phenotypes are likely to have gone undiagnosed hindering our ability to properly identify neuromuscular inflection points and reduce the detected prevalence of other phenotypes. The frequency of physician visits and the precision of disease coding by different medical professionals are not uniform, and this could lead to inaccuracies in the findings and phenotype timelines. For the temporal analysis it is possible that the proximity of neuromuscular phenotypes to SMA diagnosis could be due to their increased prevalence and how that affects the median time to event. Finally, while this study is the largest SMA study to date and only one focusing on subject health prior to neuromuscular inflection point, our limited numbers did not enable us to control for all confounding variables. However, since the intention for this study to motivate larger studies to validate and extend the findings in other pre-symptomatic cohorts, we are confident in the approach.

In conclusion, this work presents a phenome-wide analysis of patients with SMA, demonstrating association with a range of neuromuscular and non-neuromuscular phenotypes. This will be particularly important when evaluating efficacy of Spinraza compared to future systemically delivered interventions. The temporal analysis indicates that many non-neuromuscular phenotypes are present prior to early manifestations of neuromuscular degeneration. This points not only to a primary relationship of these symptoms with SMN deficiency, but also towards the possibility of their use in predicting time to neuromuscular symptom onset and treatment initiation prior to irreversible nervous system damage. Validation and extension of our findings should be performed using other existing datasets. Additionally, increased genetic screening should enable future studies of pre-symptomatic SMA patients that are capable of more in-depth uniform analyses.

### REFERENCES—EXAMPLE 2

1. Lefebvre, S., Burlet, P., Viollet, L., Bertrandy, S., Huber, C., Belser, C. and Munnich, A. A novel association of the SMN protein with two major non-ribosomal nucleolar proteins and its implication in spinal muscular atrophy. Hum Mol Genet 2002; 11: 1017-1027.
2. Lefebvre, S., Burglen, L., Frezal, J., Munnich, A. & Melki, J. The role of the SMN gene in proximal spinal muscular atrophy. Hum. Mol. Genet. 1998; 7, 1531-1536.
3. Darras B T, Monani U R, De Vivo D C. Genetic disorders affecting the motor neuron: spinal muscular atrophy. Chapter 139. In: Swaiman K F, Ashwal S, Ferriero D M, Schor N F, Finkel R S, Gropman A L, Pearl P L, Shevell M (eds). Swaiman's Pediatric Neurology: Principle and Practice. Philadelphia: Elsevier, 2017. Pp 1057-1064.
4. Hamilton, G. and Gillingwater, T. H., 2013. Spinal muscular atrophy: going beyond the motor neuron. Trends in molecular medicine, 19(1), pp. 40-50.
5. Riessland, M., Ackermann, B., Förster, A., Jakubik, M., Hauke, J., Garbes, L., Fritzsche, I., Mende, Y., Blumcke, I., Hahnen, E. and Wirth, B., 2010. SAHA ameliorates the SMA phenotype in two mouse models for spinal muscular atrophy. Human molecular genetics, 19(8), pp. 1492-1506.
6. Ottesen, E. W., Howell, M. D., Singh, N. N., Seo, J., Whitley, E. M. and Singh, R. N., 2016. Severe impairment of male reproductive organ development in a low SMN expressing mouse model of spinal muscular atrophy. Scientific reports, 6, p. 20193.
7. Richert, J. R., Antel, J. P., Canary, J. J., Maxted, W. C., Groothuis, D. Adult onset spinal muscular-atrophy with atrophic testes—report of 2 cases. Journal of Neurology Neurosurgery and Psychiatry, 1986; 49, 606-608
8. Lonsdale J, Thomas J, Salvatore M, Phillips R, Lo E, Shad S, Hasz R, Walters G, Garcia F, Young N, Foster B. The genotype-tissue expression (GTEx) project. Nature genetics. 2013 Jun. 1; 45(6):580-5.
9. Gubitz, A. K., Feng, W. and Dreyfuss, G., 2004. The SMN complex. Experimental cell research, 296(1), pp. 51-56.
10. Kolb, S. J., Battle, D. J. and Dreyfuss, G., 2007. Molecular functions of the SMN complex. Journal of child neurology, 22(8), pp. 990-994.
11. Singh, R. N., Howell, M. D., Ottesen, E. W. and Singh, N. N., 2017. Diverse role of survival motor neuron protein. Biochimica et Biophysica Acta (BBA)-Gene Regulatory Mechanisms, 1860(3), pp. 299-315.
12. Paez-Colasante, X., Seaberg, B., Martinez, T. L., Kong, L., Sumner, C. J. and Rimer, M., 2013 Improvement of neuromuscular synaptic phenotypes without enhanced survival and motor function in severe spinal muscular atrophy mice selectively rescued in motor neurons. PloS one, 8(9), p. e75866.
13. Lee, A. J., Awano, T., Park, G. H. and Monani, U. R., 2012. Limited phenotypic effects of selectively augmenting the SMN protein in the neurons of a mouse model of severe spinal muscular atrophy. PloS one, 7(9), p. e46353.
14. Wishart, T. M., Mutsaers, C. A., Riessland, M., Reimer, M. M., Hunter, G., Hannam, M. L., Eaton, S. L., Fuller, H. R., Roche, S. L., Somers, E. and Morse, R., 2014. Dysregulation of ubiquitin homeostasis and β-catenin signaling promote spinal muscular atrophy. The Journal of clinical investigation, 124(4), p. 1821.
15. Hua, Y., Liu, Y. H., Sahashi, K., Rigo, F., Bennett, C. F. and Krainer, A. R., 2015. Motor neuron cell-nonautonomous rescue of spinal muscular atrophy phenotypes in mild and severe transgenic mouse models. Genes & development, 29(3), pp. 288-297.
16. Zhou, H., Meng, J., Marrosu, E., Janghra, N., Morgan, J. and Muntoni, F., 2015. Repeated low doses of morpholino antisense oligomer: an intermediate mouse model of spinal muscular atrophy to explore the window of therapeutic response. Human molecular genetics, 24(22), pp. 6265-6277.
17. Wirth, B., Barkats, M., Martinat, C., Sendtner, M. and Gillingwater, T. H., 2015. Moving towards treatments for spinal muscular atrophy: hopes and limits. Expert Opin. Emerg. Drugs, 20 (2015), pp. 353-356
18. Tizzano, E. F. and Finkel, R. S., 2017. Spinal muscular atrophy: A changing phenotype beyond the clinical trials. Neuromuscular Disorders, 27(10), pp. 883-889.
19. Luo, J., Wu, M., Gopukumar, D. and Zhao, Y., 2016. Big data application in biomedical research and health care: A literature review. Biomedical informatics insights, 8, p. 1.
20. Goldstein, B. A., Navar, A. M., Pencina, M. J. and Ioannidis, J., 2017. Opportunities and challenges in developing risk prediction models with electronic health records data: a systematic review. Journal of the American Medical Informatics Association, 24(1), pp. 198-208.
21. Miotto, R., Wang, F., Wang, S., Jiang, X. and Dudley, J. T., 2017. Deep learning for healthcare: review, opportunities and challenges. Briefings in Bioinformatics, p. bbx044.
22. Armstrong, E. P., Malone, D. C., Yeh, W., Dahl, G. J., Lee, R. and Sicignano, N., 2015. The economic burden of Spinal Muscular Atrophy. Value in Health, 18(3), p. A282.
23. Kolb, S. J. and Kissel, J. T., 2011. Spinal muscular atrophy: a timely review. Archives of neurology, 68(8), pp. 979-984.
24. Denny, J. C., Bastarache, L., Ritchie, M. D., Carroll, R. J., Zink, R., Mosley, J. D., Field, J. R., Pulley, J. M., Ramirez, A. H., Bowton, E. and Basford, M. A., 2013. Systematic comparison of phenome-wide association study of electronic medical record data and genome-wide association study data. Nature biotechnology, 31(12), p. 1102.
25. R Core Team (2015). R: A language and environment for statistical computing. R Foundation for Statistical Computing, Vienna, Austria. URL https://www.R-project.org/.
26. Benjamini, Y. and Hochberg, Y. Controlling the false discovery rate: a practical and powerful approach to multiple testing. Journal of the royal statistical society. Series B (Methodological). 1995; pp. 289-300.
27. Zerres, K. and Rudnik-Schoneborn, S., 1995. Natural history in proximal spinal muscular atrophy: clinical analysis of 445 patients and suggestions for a modification of existing classifications. Archives of neurology, 52(5), pp. 518-523.
28. Zerres, K., Rudnik-Schoneborn, S., Forrest, E., Lusakowska, A., Borkowska, J. and Hausmanowa-Petrusewicz, I., 1997. A collaborative study on the natural history of childhood and juvenile onset proximal spinal muscular atrophy (type II and III SMA): 569 patients. Journal of the neurological sciences, 146(1), pp. 67-72.
29. Kaufmann, P., McDermott, M. P., Darras, B. T., Finkel, R. S., Sproule, D. M., Kang, P. B., Oskoui, M., Constantinescu, A., Gooch, C. L., Foley, A. R. and Yang, M. L., 2012. Prospective cohort study of spinal muscular atrophy types 2 and 3. Neurology, 79(18), pp. 1889-1897.
30. Bertini, E. and Mercuri, E., 2018. Motor neuron disease: A prospective natural history study of type 1 spinal muscular atrophy. Nature Reviews Neurology.
31. Hsieh-Li, H. M., Chang, J. G., Jong, Y. J., Wu, M. H., Wang, N. M., Tsai, C. H. and Li, H., 2000. A mouse model for spinal muscular atrophy. Nature genetics, 24(1), p. 66.
32. Narver, H. L., Kong, L., Burnett, B. G., Choe, D. W., Bosch-Marcé, M., Taye, A. A., Eckhaus, M. A. and Sumner, C. J., 2008. Sustained improvement of spinal muscular atrophy mice treated with trichostatin A plus nutrition. Annals of neurology, 64(4), pp. 465-470.
33. Bevan, A. K., Hutchinson, K. R., Foust, K. D., Braun, L., McGovern, V. L., Schmelzer, L., Ward, J. G., Petruska, J. C., Lucchesi, P. A., Burghes, A. H. and Kaspar, B. K., 2010. Early heart failure in the SMNA7 model of spinal muscular atrophy and correction by postnatal scAAV9-SMN delivery. Human molecular genetics, 19(20), pp. 3895-3905.
34. Heier, C. R., Satta, R., Lutz, C., and DiDonato, C. J. Arrhythmia and cardiac defects are a feature of spinal muscular atrophy model mice. Hum Mol Genet, 2010; 19, 3906-3918.
35. Shababi, M., Habibi, J., Yang, H. T., Vale, S. M., Sewell, W. A., and Lorson, C. L. Cardiac defects contribute to the pathology of spinal muscular atrophy models. Hum Mol Genet, 2010; 19, 4059-4071.
36. Schreml, J., Riessland, M., Paterno, M., Garbes, L., RoBbach, K., Ackermann, B., Kramer, J., Somers, E., Parson, S. H., Heller, R. and Berkessel, A. Severe SMA mice show organ impairment that cannot be rescued by therapy with the HDACi JNJ-26481585. European Journal of Human Genetics, 2013; 21(6), pp. 643-652.
37. Richert, J. R., Antel, J. P., Canary, J. J., Maxted, W. C., Groothuis, D. Adult onset spinal muscular-atrophy with atrophic testes—report of 2 cases. Journal of Neurology Neurosurgery and Psychiatry, 1986; 49, 606-608

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them Implementations of the subject matter described in this specification can be implemented as one or more computer programs, i.e., one or more modules of computer program instructions, encoded on computer storage medium for execution by, or to control the operation of, data processing apparatus.

In this respect, it should be recognized that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be recognized that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### CONCLUSION

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A method for identifying an agent that corrects a phenotype associated with a condition, comprising:
receiving, at a processor, a set of structured health data comprising electronic health records of a set of patients each comprising at least one diagnostic code associated with the condition;
processing, by the processor, the set of structured health data using a first algorithm to output a group of phenotypic clusters wherein each of the phenotypic clusters comprises a sub-set of the set of patients;
receiving, at the processor, genetic data for the set of patients;
processing, by the processor, the genetic data for a first phenotypic cluster of the group of phenotypic clusters using a second algorithm to output a set of phenotypic-genomic sub-clusters, wherein each of the phenotypic-genetic sub-cluster represents a subset of the patients of the first phenotypic cluster;
isolating cells from each of the patients in a first phenotypic-genomic sub-cluster of the set of phenotypic-genomic sub-clusters;
differentiating the cells into one or more disease-affected cell types;
assaying the disease-affected cells to identify a first disease phenotype associated with the condition;
contacting the disease-affected cells with a candidate agent;
assaying the disease-affected cells for the first disease phenotype after contacting the disease-affected cells with the candidate agent to determine whether the candidate agent corrected the phenotype; and
identifying the candidate agent as a treatment for the first phenotypic-genomic sub-cluster if the candidate agent corrected the phenotype.

2. The method of claim 1, wherein the st differentiating the cells into all three germ lines simultaneously or individually using directed differentiating techniques.

3. The method of claim 2 or any other preceding claim, wherein the first disease phenotype is in germ layers differentiated into non-neural tissue and wherein the poly-genetic condition is a neuropsychiatric disorder.

4. The method of claim 1 or any other preceding claim, wherein the set of structured health data comprises diagnostic codes from electronic medical records.

5. The method of claim 4 or any other preceding claim, wherein the first algorithm comprises aggregating the diagnostic codes into a set of categories using data from the phenotype-side association study (PheWAS).

6. The method of claim 5 or any other preceding claim, further comprising processing the diagnostic codes into dimensional vectors describing the counts of the most common diagnostic codes in specific time windows for the patients.

7. The method of claim 5 or any other preceding claim, wherein the first algorithm comprises hierarchical clustering.

8. The method of claim 7 or any other preceding claim, wherein hierarchical clustering is performed using Euclidean distance and Ward's method.

9. The method of claim 1 or any other preceding claim, wherein assaying the cells comprises using at least one of the following assays: image-based assays examining cell properties comprising proliferation, differentiation or migration, cell death markers or oxidative stress dyes, RNA sequencing (bulk or single cell), or electrophysiology testing.

10. The method of claim 5 or any other preceding claim, wherein the step of differentiating the cells comprises differentiating the cells into tissue types related to the set of categories.

11. The method of claim 1 or any other preceding claim, wherein the genetic data comprises mutations related to the condition.

12. The method of claim 1 or any other prece [symbol] comprises a clustering algorithm.

13. A method for identifying an agent that corrects a phenotype associated with a condition, comprising:
receiving, a set of phenotypic-genomic sub-clusters that were output from a processor that had performed the following steps:
receiving, at the processor, a set of structured health data comprising electronic health records of a set of patients each comprising at least one diagnostic code associated with the condition;
processing, by the processor, the set of structured health data using a first algorithm to output a group of phenotypic clusters wherein each of the phenotypic clusters comprises a sub-set of the set of patients;
receiving, at the processor, genetic data for the set of patients;
processing, by the processor, the genetic data for a first phenotypic cluster of the group of phenotypic clusters using a second algorithm to output a set of phenotypic-genomic sub-clusters, wherein each of the phenotypic-genetic sub-cluster represents a subset of the patients of the first phenotypic cluster; and
isolating cells from each of the patients in a first phenotypic-genomic sub-cluster of the set of phenotypic-genomic sub-clusters;
differentiating the cells into one or more disease-affected cell types;
assaying the disease-affected cells to identify a first disease phenotype associated with the condition;
contacting the disease-affected cells with a candidate agent;
assaying the disease-affected cells for the first disease phenotype after contacting the disease-affected cells with the candidate agent to determine whether the candidate agent corrected the phenotype; and
identifying the candidate agent as a treat [symbol] cluster if the candidate agent corrected the phenotype.

14. A method of identifying whether a medical record of a patient indicates a patient is likely to develop a genetic disease, the method comprising:
receiving, at a processor, a set of structured health data comprising an electronic health record of a patient comprising at least one diagnostic code associated with the genetic condition, a set of additional diagnostic codes, and genetic data;
processing, at the processor, the set of structured health data to determine whether the patient is a match for a phenotypic-genetic sub-cluster; and
flagging, by the process, the patient record to indicate the patient is likely to develop the disease within a certain time window.

15. The method of claim 14, wherein the processor re-determines whether the patient is a match for a phenotypic-genetic sub-cluster each time the patient's electronic health record receives a new diagnostic code.

16. The method of claim 14, further comprising treating the patient with a drug associated with the phenotypic-genetic sub-cluster as treatment.

17. The method of claim 14, wherein the disease is a CNS disorder.

18. The method of claim 14, wherein the disease is Spinal Muscular Atrophy.

19. The method of claim 14, wherein the step of determining whether the patient is a match for a phenotypic-genetic sub-cluster further comprises:
isolating cells from the patient;
differentiating the cells into one or more disease-affected cell types;
assaying the disease-affected cells to identify a first disease phenotype associated with the condition; and
determining whether the patient is a match for the phenotypic-genetic sub-cluster based on the assays.

20. A method of preventing the onset of a di [symbol] comprising:
receiving, at a processor, a set of structured health data comprising an electronic health record of a patient comprising at least one diagnostic code associated with the genetic condition, a set of additional diagnostic codes, and genetic data;
processing, at the processor, the set of structured health data to determine whether the patient is a match for a phenotypic-genetic sub-cluster;
flagging, by the process, the patient record to indicate the patient is likely to develop the disease within a certain time window;
isolating cells from the patient;
differentiating the cells into one or more disease-affected cell types;
assaying the disease-affected cells to identify a first disease phenotype associated with the condition;
contacting the disease-affected cells with a candidate agent;
assaying the disease-affected cells for the first disease phenotype after contacting the disease-affected cells with the candidate agent to determine whether the candidate agent corrected the phenotype;
identifying the candidate agent as a treatment for the first phenotypic-genomic sub-cluster if the candidate agent corrected the phenotype; and
administering the candidate agent to the subject.

21. The method of claim 20, wherein the patient does not exhibit a common symptom of the disease or disorder prior to administration.

22. The method of claim 20 or any other preceding claim, wherein administration treats the disease or disorder.

23. The method of claim 20 or any other preceding claim, wherein administration treats the first disease phenotype associated with the disease or disorder.

24. The method of claim 22 or 23, wherein tre [symbol] second symptom associated with the disease or disorder.

25. The method of any one of the preceding claims, wherein the cells from the patient are stem cells.

26. The method of claim 25, wherein the stem cells are selected from embryonic stem cells, adult stem cells, or cord blood stem cells.

27. The method of any one of the preceding claims, wherein the cells from the patient are somatic cells.

28. The method of claim 27, wherein the somatic cells are fibroblasts.

29. The method of claim 27 or 28, wherein the method further comprises reprogramming the cells into induced pluripotent stem (iPS) cells, and then differentiating the iPS cells into the one or more disease-affected cell types.
